SEQUENCE LISTING (4 sequences)

SEQ ID NO: 1 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 2 - protein, 385 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCKNPGSRAGNSAHQPPHPQPPVRFHPPLRRAGKVP

SEQ ID NO: 3 - protein, 328 aa, Mus musculus
SLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 4 - protein, 417 aa, Hydra vulgaris
MQYKLALNGKTLKGETTTEAVDAATAEKVFKQYANDNGVDGEWTYDDATKTFTVTELVPRGSQLWMALGTQTSAIESRPRSSINKNLCRALYLHHYQRTVCLNYTDLMLSVAEGIRLGIDECQVQFKHRKWNCTINEHGTSVFGPIITTASRESAFISGIISAGVAFSVTESCAEGKSVHCRCDNSVRGQTDEGWRWGGCNRPITYGIWFSQLFIDQVEKIVKKRKDPRKIMNLHNNKAGREVIKNLLQTECKCHGTSGNCNLKTCWRSQPHFSEIGKILKEKYDSAHEMEFLYKVKANGERKIKDLIPKYKEYLPPSSLDFIYYEESPNYCVKNETLGIAGTKGRSCNITSSGVDGCELMCCQRGYNVNIVQKTHSCECKFVWCCKVSCNSCIKMTPEYTCKLVPRGSLEHHHHHH

(19) United States
(12) Patent Application Publication
(10) Pub. No.: US 2014/0302111 A1
(43) Pub. Date: Oct. 9, 2014

(54) COMPOSITIONS AND METHODS FOR DENTAL TISSUE REGENERATION

(71) Applicants: Jeremy J. Mao, Closter, NJ (US); Ying Zheng, Fort Lee, NJ (US); Guoming Sun, New York, NY (US); Songlin Wang, Beijing (CN)

(72) Inventors: Jeremy J. Mao, Closter, NJ (US); Ying Zheng, Fort Lee, NJ (US); Guoming Sun, New York, NY (US); Songlin Wang, Beijing (CN)

(73) Assignee: The Trustees of Columbia University in the City of New York, New York, NY (US)

(21) Appl. No.: 14/222,526

(22) Filed: Mar. 21, 2014

Related U.S. Application Data
(63) Continuation-in-part of application No. 12/739,902, filed on Oct. 20, 2010, now abandoned.
(60) Provisional application No. 61/041,681, filed on Apr. 2, 2008.
(60) Provisional application No. 60/982,671, filed on Oct. 25, 2007.
(60) Provisional application No. 61/831,595, filed on Jun. 5, 2013.
(60) Provisional application No. 61/804,138, filed on Mar. 21, 2013.

(51) Int. Cl. A61L 26/00; A61K 38/18; A61K 38/17

(52) U.S. Cl. CPC A61L 26/0061; A61K 38/1709; A61K 38/1875; A61K 38/18; A61K 38/1825; A61K 38/185
USPC 424/423; 514/8.8; 514/8.1; 514/8.4

(57) ABSTRACT

Provided is a method for regenerating dental tissue, which can include contacting a scaffold containing Wnt3a and BMP-7, and optionally VEGF, bFGF, or NGF, with a dental tissue so as to promote odontoblastic differentiation of a progenitor cell, promote progenitor cell migration into the dental tissue, or regenerate the dental tissue. Also provided is a composition for regeneration of dental tissue, which can include a scaffold and Wnt3a and BMP-7, and optionally VEGF, bFGF, or NGF.

### CROSS-REFERENCE TO RELATED APPLICATIONS

The present application is a: (1) U.S. Continuation-In-Part of U.S. National Phase application Ser. No. 12/739,902 filed 23 Oct. 2008, which is a 371 of International Application No. PCT/US08/81011 filed 23 Oct. 2008, which claims the benefit of U.S. Provisional Application No. 61/041,681 filed 2 Apr. 2008 and U.S. Provisional Application No. 60/982,671 filed 25 Oct. 2007; and (2) claims the benefit of priority to U.S. Provisional Application No. 61/804,138 filed 21 Mar. 2013 and U.S. Provisional Application No. 61/831,595 filed 5 Jun. 2013; each of which is incorporated herein by reference in its entirety.

### STATEMENT REGARDING FEDERALLY SPONSORED RESEARCH OR DEVELOPMENT

This invention was made with government support under Grant No. 5RC2DE020767 awarded by National Institutes of Health/National Institute of Dental and Craniofacial Research and under Grant No. R01DE15391 awarded by The National Institutes of Health. The government has certain rights in the invention.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### BACKGROUND OF THE INVENTION

The tooth is biologically viable largely because of the tooth pulp. Currently, diseased, missing or traumatized dental pulp is treated by capping or replacement with inert synthetic materials. The most common filling material is gutta-percha, a thermoplastic polymer of isoprene. After removal of the native tooth pulp that has been diseased, is missing or is traumatized, gutta-percha is melted and injected to fill the root canal. Although endodontic or root canal treatment has been the conventional state of art of contemporary dentistry, it has several deficiencies that negatively affect the quality of life of the patient (Salvi et al. 2007). First, root canal-treated teeth tend to be brittle, and susceptible to fracture. Second, discoloration frequently takes place following root canal treatment. Patients whose root canal treated teeth have undergone discoloration often require additional and costly cosmetic dental procedures. Third, diseased, missing or infected tooth pulp of deciduous (baby) teeth often lacks treatment options and is frequently not suitable for root canal treatment. Pulp necrosis happens in 85-96% of the avulsed teeth and in 70-100% of the intruded teeth. Untreated or poorly managed dental infections may be the causes of systemic infections (Shay 2002; Brennan et al. 2007).

Ideally, an improved treatment for teeth having diseased, missing or traumatized pulp causes the restoration of biologically vital tissue. Tissue engineering techniques have been used in the development of methods and compositions for restoring craniofacial tissues and bone. See, e.g., Alhadlaq and Mao, 2003; Edwards and Mason, 2006; Fong et al., 2005; Goldberg and Smith, 2004; Hong and Mao, 2004; Lovschall et al., 2001; Mao et al., 2006; Mathieu et al., 2006; Murray et al., 2002; Murray et al., 2007; Nakashima and Alamine, 2005; Nakashima and Reddi, 2003; Stosich and Mao, 2007; Young et al., 2002; U.S. Pat. No. 5,885,829; and U.S. Patent Application Publication 20050079470. Most of those techniques involve the use of scaffolding materials that comprise mammalian cells such as dental pulp stem cells or mesenchymal stem cells, and/or bioactive ingredients such as bone morphogenic proteins (BMP). Techniques where cells are seeded onto the scaffolding material have the disadvantage of being difficult to prepare and store, since viable cells must be seeded, cultured and maintained on the scaffolding. Additionally, the source and yield of cells used in the regeneration of tissues can be inadequate.

Pivotal factors that induce progenitor cells into odontoblasts (Od) are unknown.

### SUMMARY OF THE INVENTION

Among the various aspects of the present disclosure is the provision of compositions and methods for regenerating dental tissue.

One aspect provides a composition for regenerating dental tissue. In some embodiments, the composition includes a matrix or scaffold (e.g., a matrix or scaffold comprising a hydrogel) and a therapeutically effective amount of Wnt3a polypeptide and bone morphogenetic protein 7 (BMP-7). In some embodiments, the composition includes a therapeutically effective amount of vascular endothelial growth factor (VEGF), basic fibroblast growth factor (bFGF), or nerve growth factor (NGF). In some embodiments, the composition does not comprise a living cell. In some embodiments, the matrix or scaffold comprises Wnt3a and BMP-7 and, optionally, VEGF, bFGF, or NGF. Other features of the composition are discussed below in the context of methods of use. One of ordinary skill will understand that such features can equally apply to the composition itself.

Another aspect provides a method for regenerating dental tissue. In some embodiments, the method includes providing a composition described above and inserting the composition into a natural or artificial cavity or chamber of a mammalian tooth. In some embodiments, the composition promotes odontoblastic differentiation of a progenitor cell, promotes progenitor cell migration into the dental tissue, promotes angiogenic, odontogenic, fibrogenic, or neurogenic development, so as to regenerate the dental tissue. In some embodiments, the composition regenerates vascularized pulp tissue or regenerates neodentin.

In some embodiments, the scaffold further includes a compound selected from the group consisting of platelet-derived growth factor (PDGF), endothelial cell growth factor (ECGF), transforming growth factor-β1 (TGF-β1), epidermal growth factor (EGF), hepatocyte growth factor (HGF), stromal cell-derived factor-1 (SDF1), a bone morphogenetic protein (BMP) other than BMP-7, a TGF-β, a growth and differentiation factor (GDF), insulin-like growth factor-1 (IGF1), a dentin matrix protein, a dentin sialoprotein, a bone sialoprotein, amelogenin, an integrin; an angiogenin; an angiopoietin-1; a del-1; a follistatin; a granulocyte colony-stimulating factor (G-CSF); a hepatocyte growth factor/scatter factor (HGF/SF); an interleukin-8 (IL-8); a leptin; a midkine; a placental growth factor; a platelet-derived endothelial cell growth factor (PD-ECGF); a platelet-derived growth factor-BB (PDGF-BB); a pleiotrophin (PTN); a progranulin; a proliferin; a transforming growth factor-α (TGF-α); a transforming growth factor-β (TGF-β); a tumor necrosis factor-α (TNF-α); a matrix metalloproteinase (MMP); an angiopoietin 1 (ang1); an ang2; a delta-like ligand 4 (DLL4); a connective tissue growth factor (CTGF); a brain derived nerve factor (BDNF); an NT-4; and an NT-3. In some embodiments, the scaffold further comprises an antibiotic or an analgesic.

In some embodiments, the Wnt3a polypeptide, BMP-7, VEGF, bFGF, or NGF is injected into, mixed into, encapsulated in, tethered to, or absorbed in the matrix or scaffold.

In some embodiments, the scaffold has the shape of a human incisor, a human cuspid, a human bicuspid or a human molar, or a natural or artificial cavity or chamber therein.

In some embodiments, the matrix or scaffold includes a natural polymer selected from the group consisting of collagen, gelatin, polysaccharide, chitosan, hydroxyapatite (HA), and polyhydroxyalkanoate. In some embodiments, the matrix or scaffold includes a synthetic polymer selected from the group consisting of an aliphatic polyester of a poly(α-hydroxy acid), and a polyethylene glycol. In some embodiments, the matrix or scaffold includes hydroxyapatite. In some embodiments, the matrix or scaffold includes an alginate hydrogel. In some embodiments, the matrix or scaffold is biodegradable.

In some embodiments, the scaffold includes microchannels having a diameter of (i) between 50 and 500 μm; or (ii) about 200 μm. In some embodiments, the Wnt3a and BMP-7 and, optionally, VEGF, bFGF, or NGF, are imbedded in a gel in the microchannels of the scaffold. In some embodiments, the Wnt3a and BMP-7 and, optionally, VEGF, bFGF, or NGF, are encapsulated in a microsphere.

In some embodiments, the method further includes making a model of a tooth or tooth cavity using computer aided design (CAD) and synthesizing the scaffold with a bioplotter.

In some embodiments, the Wnt3a polypeptide includes an amino acid sequence of SEQ ID NO: 1, SEQ ID NO: 2, SEQ ID NO: 3, or SEQ ID NO: 4. In some embodiments, the Wnt3a polypeptide includes an amino acid sequence having at least about 95% sequence identity to SEQ ID NO: 1, SEQ ID NO: 2, SEQ ID NO: 3, or SEQ ID NO: 4 and Wnt3a activity.

In some embodiments, Wnt3a is present at a concentration of about 1 ng to about 1,000 mg Wnt3a per ml of solution, where the solution is introduced into the matrix or scaffold. In some embodiments, BMP-7 is present at a concentration of about 1 ng to about 1,000 mg BMP-7 per ml of solution, where the solution is introduced into the matrix or scaffold. In some embodiments, Wnt3a is present at a concentration of about 10 ng per ml of solution and BMP-7 is present at a concentration of about 200 ng per ml of solution, where the solution is introduced into the matrix or scaffold. In some embodiments, VEGF is present at a concentration of about 1 ng to about 1,000 mg VEGF per ml of solution. In some embodiments, bFGF is present at a concentration of about 1 ng to about 1,000 mg bFGF per ml of solution. In some embodiments, NGF is present at a concentration of about 1 ng to about 1,000 mg NGF per ml of solution. In some embodiments, a solution described above is introduced into the matrix or scaffold.

In some embodiments, the method further includes exposing traumatized or diseased dental pulp tissue in the tooth pulp chamber or root canal; and capping or filling at least a portion of a tooth pulp chamber or a root canal with the composition, wherein de novo vascularized pulp-like tissue forms in the tooth pulp chamber or root canal after capping or filling.

In some embodiments, the method further includes removing traumatized or diseased dental pulp tissue from the tooth to create a tooth pulp chamber or root canal substantially devoid of traumatized or diseased tissue. In some embodiments, the method further includes removing substantially all dental pulp tissue from the tooth. In some embodiments, the method further includes filling at least a portion of the tooth pulp chamber with an inert material.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-C are a series of photographs of adult human teeth that underwent clinically equivalent root canal treatment.

FIG. 1A shows the endodontically treated root canal and pulp chamber filled with collagen sponge without a bioactive ingredient.

FIG. 1B shows the endodontically treated root canal and pulp chamber with basic fibroblast bioactive ingredient (bFGF) only.

FIG. 1C shows the both bFGF and vascular endothelial bioactive ingredient (VEGF). The teeth were implanted subcutaneously in immunodeficient mice for 2 weeks to evaluate whether vascularization takes place in the endodontically treated root canal and pulp chamber. As opposed to the no bioactive ingredient treatment (collagen sponge only) (FIG. 1A), bFGF only (FIG. 1B) and VEGF and bFGF combined (FIG. 1C) both showed vascularization in the collagen sponge inserted in the root canal.

FIG. 2A-D shows micrographs of sections of adult human teeth treated as in FIG. 1A-C.

FIG. 2A shows the root canal of a permanent human incisor with an implanted collagen sponge without a bioactive ingredient. There is a lack of any host tissue ingrowth from apical foramen following in vivo implantation in immunodeficient mice.

FIG. 2B shows a root canal of a permanent human incisor with a VEGF-loaded collagen sponge, showing the presence of vascularization (arrow), and host tissue ingrowth. The infiltrating host tissue is attached to the dentin.

FIG. 2C shows a root canal of a permanent human incisor with a bFGF-loaded collagen sponge, showing the presence of vascularization (arrow), and host tissue ingrowth. The infiltrating host tissue is attached to the dentin.

FIG. 2D shows a root canal of a permanent human incisor with a VEGF+bFGF-loaded collagen sponge showing the presence of vascularization (arrow), and host tissue ingrowth. The infiltrating host tissue is attached to the dentin.

FIG. 3 is a graph showing the release kinetics of TGFβ3 from PLGA microspheres in a 1% BSA solution. TGFβ3 was released in a sustained fashion up to 36 and 42 days from 50:50 or 75:25 co-polymer ratios of PLGA microspheres, respectively, as detected by ELISA. Initial burst-like release was observed for both co-polymer ratios, although the 50:50 PLA/PGA ratio yielded a more rapid release rate than the 75:25 PLA/PGA ratio did.

FIG. 4A-B are scanning electron micrographs showing the fabrication and degradation of PLGA microspheres.

FIG. 4A shows a representative SEM image of microspheres fabricated from poly-d-l-lactic-co-glycolic acid (PLGA) with 50:50 PLA/PGA ratio with encapsulated TGFβ3.

FIG. 4B shows a representative SEM image of the anticipated degradation of TGFβ3 encapsulating PLGA microspheres in PBS solution.

FIG. 5 is a graph showing the cumulative average release of BMP-7 from PLGA microspheres.

FIG. 6 is a graph showing the cumulative average release of NGF from PLGA microspheres.

FIG. 7A-U are a series of images, a scatter plot, and an array diagram characterizing reporter gene expression in col1a1 (2.3 kb)-EGFP mice and microarray analysis of odontoblast and osteoblast.

FIG. 7A is a diagram of the transgene, which contains the 2.3 kb col1a1 promoter driving EGFP expression.

FIG. 7B is an image of jaw bone isolated from transgenic mice, where GFP signal can be detected in both bone and tooth.

FIG. 7C shows calvaria of Col1a1 mouse that was dissected and the expression of transgene observed in suture and bone.

FIG. 7D shows a section of jaw bone and molar.

FIG. 7E shows GFP signal was observed in alveolar bone, periodontal ligament and odontoblast.

FIG. 7F shows that in incisor of GFP positive mice, the GFP positive cells were lined up along dentin surface and GFP signal locates in odontoblasts.

FIG. 7G shows that in incisor of GFP positive mice, the GFP positive cells were lined up along dentin surface and GFP signal locates in odontoblasts.

FIG. 7H shows GFP positive odontoblasts form dentinal tubules.

FIG. 7I shows GFP positive odotoblasts migrated out from dental pulp of Col1a1-EGFP mice.

FIG. 7J shows 10,000 GFP positive cells (sorted on GFP) from dental pulp seeded subcutaneously in collagen gel (0.1 ml). X-ray image inset at left corner of FIG. 7J shows 3 weeks later, mineralized tissue was formed.

FIG. 7K shows mineralized tissue was GFP positive.

FIG. 7L is a tissue section showing that the delivered GFP positive odontoblasts formed mineralized structure similar to disorganized dentin.

FIG. 7M is an image showing dentin-like tissue positive for DSP (green: GFP, red: DSP, purple, DAPI).

FIG. 7N shows 10,000 GFP positive cells (sorted on GFP) from dental pulp implanted into renal capsule (0.1 ml). X-ray image inset at left corner of FIG. 7N shows 3 weeks later, mineralized tissue was formed.

FIG. 7O shows H&E staining of mineralized tissue.

FIG. 7P shows cells formed a mineralized tissue with DSPP positive.

FIG. 7Q shows osteocytes and osteoblasts of calvaria were GFP positive in the Col1a1-EGFP mice.

FIG. 7R shows osteocytes and osteoblasts of calvaria were GFP positive in the Col1a1-EGFP mice.

FIG. 7S shows cells isolated from calvaria bone and cultured in OIM, where GFP cells form a nodule in one week.

FIG. 7T and FIG. 7U show Agilent whole genome microarray analysis for mRNA isolated from GFP positive cells from tooth (odontoblasts) and calvaria bone (osteoblast).

FIG. 7T shows a scatter plot of signal intensities of all spots.

FIG. 7U shows an example of the data of one array experiment (n=4). The signal intensities of each feature are represented by a dot.

FIG. 8A-J are a series of images showing differentially expressed gene by immunohistochemistry and in situ hybridization. Immunohistochemistry were performed to test the protein level of selected genes. (Scale bar: A-G; 50 μm, H, I, J: 250 μm) (De: dentin, P: pulp).

FIG. 8A shows expression of selected genes which are highly expressed in odontoblasts in incisor, molar and calvaria bone.

FIG. 8B shows expression of selected genes which are highly expressed in odontoblasts in incisor, molar and calvaria bone.

FIG. 8C shows expression of selected genes which are highly expressed in odontoblasts in incisor, molar and calvaria bone.

FIG. 8D shows expression of selected genes which are highly expressed in odontoblasts in incisor, molar and calvaria bone.

FIG. 8E shows expression of selected genes which are highly expressed in odontoblasts in incisor, molar and calvaria bone.

FIG. 8F shows expression of selected genes which are highly expressed in osteoblasts. (scale bar=20 μm).

FIG. 8G shows expression of selected genes which are highly expressed in osteoblasts. (scale bar=20 μm)

FIG. 8H shows expression of the growth factors examined by in situ hybridization, because Wnts and BMP7 are secreted growth factors which may diffuse to the surrounding tissue.

FIG. 8I shows expression of the growth factors examined by in situ hybridization, because Wnts and BMP7 are secreted growth factors which may diffuse to the surrounding tissue.

FIG. 8J show expression of the growth factors examined by in situ hybridization, because Wnts and BMP7 are secreted growth factors which may diffuse to the surrounding tissue.

FIG. 9A-B are a series of bar graphs showing confirmation of the microarray by RT-PCR. The expression of the selected genes was confirmed by real time PCR using RNA isolated from GFP positive odontoblasts and osteoblasts (n=3). The genes selected are growth or secreted factors and transcription factors.

FIG. 9A shows selected genes highly expressed in odontoblasts.

FIG. 9B shows selected genes highly expressed in osteoblasts.

FIG. 10A-D are a series of bar graphs and an image showing Wnt3a selectively induces the migration of dentinogenic cells.

FIG. 10A is a bar graph showing results of a Boyden chamber assay performed using PDL cells to study the cells migration in response of Wnt3a, BMP-7. 100 k PDL cells were seeded in the well and the migration cues were added to the chamber. 12 hours later, the migrated cells were trypsinized and counted. Then the cells were plated and propagated for further studies (n=4 *p<0.05).

FIG. 10B is an image of migrated cells differentiated in osteogenic induction medium and 3 weeks after, stained with silver nitrate (von Kossa).

FIG. 10C is a bar graph showing the expression of marker genes tested by PCR. (n=3 *p<0.05).

FIG. 10D is a bar graph showing the expression of marker genes tested by PCR. (n=3 *p<0.05).

FIG. 11 is a pair of images showing shows dental pulp of minipig removed through the standard pulp ectomy procedure. Access openings were made into the pulp chambers. After extirpation of the dental pulp tissue, the pulp chamber was cleaned. Root canal was shaped with hand files ranging in a diameter from 0.1 to 2.5 mm and washing with saline. The canal was dried with sterile paper points.

FIG. 12A-L are a series of images and bar graphs showing Wnt3a or BMP-7 regenerated dental pulp and dentin.

FIG. 12A shows injectable collagen gel injected into the root canal. The open access cavity was sealed with basing material and composite resin.

FIG. 12B shows injectable collagen gel combined with growth factor, BMP-7, injected into the root canal. The open access cavity was sealed with basing material and composite resin.

FIG. 12C shows injectable collagen gel combined with growth factor, Wnt 3a, injected into the root canal. The open access cavity was sealed with basing material and composite resin.

FIG. 12D shows injectable collagen gel combined with growth factors, BMP-7 and Wnt 3a, injected into the root canal. The open access cavity was sealed with basing material and composite resin.

FIG. 12E is an enlargement of a portion of FIG. 12D.

FIG. 12F is an enlargement of a portion of FIG. 12D.

FIG. 12G shows the incisors harvested and examined after 6-8 weeks with histology and micro-CT.

FIG. 12H shows the incisors harvested and examined after 6-8 weeks with histology and micro-CT.

FIG. 12I shows the incisors harvested and examined after 6-8 weeks with histology and micro-CT.

FIG. 12J shows the incisors harvested and examined after 6-8 weeks with histology and micro-CT.

FIG. 12K shows quantities of newly formed dentin volume and density (p<0.05, n=4).

FIG. 12L shows quantities of newly formed dentin volume and density (p<0.05, n=4).

FIG. 13A-D are a series of drawings depicting dental pulp decompression filling membrane where: 1 is the membrane; 2 is the periphery attachment of composite; 3 is the prepared tooth cavity; 4 is the absorbable biomaterial; 5 is the connection of microporous membrane and tooth cavity; and 6 is the pulp tissue.

FIG. 13A shows a top view of the filling membrane showing the center part of the membrane and the peripheral part of the composite.

FIG. 13B shows a sectional view of the filling membrane showing the membrane carried in the composite.

FIG. 13C shows a sectional view of the sealing device with the tooth chamber, shown in this perspective in place. The exudation of inflammation in the tooth chamber can be absorbed instantly by the alginate and subsequently infiltrate from the membrane while the bacteria in oral cavity is prevented by the membrane.

FIG. 13D shows a sectional view of the sealing device with the tooth.

FIG. 14 is a reaction scheme of the formation of alginate hydrogel.

FIG. 15 is a bar graph of the swelling ratio (%) of physically cross linked alginate hydrogels.

FIG. 16 is a photograph of the mouth of an anesthetized minipig, where a rubber dam was placed to isolate incisors. The pulp chamber was accessed by mechanical opening with high speed bur.

FIG. 17 is an image of a tissue cross section of an incisor of a minipig showing regenerated pulp (r.p.), regenerated blood vessels (b.v.), regenerated dentin (r.d.), and native denton (n.d.) formed four (4) weeks after hydrogel with BMP-7 (200 ng/ml) was injected into the root canal and pulp chamber.

FIG. 18 is an image of a tissue cross section of an incisor of a minipig showing regenerated pulp (r.p.), regenerated blood vessels (b.v.), regenerated dentin (r.d.), and native denton (n.d.) formed four (4) weeks after hydrogel with Wnt protein (10 ng/ml) was injected into the root canal and pulp chamber.

FIG. 19 is an image of a tissue cross section of an incisor of a minipig showing regenerated pulp (r.p.), regenerated blood vessels (b.v.), regenerated dentin (r.d.), and native denton (n.d.) formed four (4) weeks after hydrogel with BMP-7 (200 ng/ml) and Wnt protein (10 ng/ml) was injected into the root canal and pulp chamber.

### DETAILED DESCRIPTION OF THE INVENTION

The present disclosure is based, at least in part, on the discovery that diseased, traumatized or missing tooth pulp can be replaced with a composition comprising a matrix or scaffold and one or more bioactive ingredients that promotes angiogenic, odontogenic, fibrogenic, or neurogenic development. Such a composition can promote angiogenic, odontogenic, fibrogenic or neurogenic development into the pulp chamber, preserving the vitality of the tooth.

Provided herein are compositions including one or more bioactive agents for regeneration of dental pulp or dentin. Various compositions can include bioactive agents such as Wnt3a, BMP-7, VEGF, bFGF, NGF, or combinations thereof.

Also provided are several biocompatible materials to preserve the vitality of dental pulp in the stage of reversible pulpitis. As shown herein, several resorbable or irresorbable biomaterials, including porous membrane and hydrogels, not only allow the diffusion of blood, tissue fluid or water, but can also prevent pathogens in the oral cavity to enter pulp tissue. Thus, dental pulp in reversible pulpitis can be saved to maintain its vitality.

The present disclosure is based, at least in part, on the discovery of key factors for the specification of odontoblasts from a progenitor cell, such as a stem cell, by global gene expression profiles. These pivotal factors were used in an in vivo preclinical dental pulp and dentin model. As shown herein, these pivotal factors, including Wnt proteins, induced robust regeneration of not only dental pulp, but also dentin. Delivery of these pivotal factors can replace root canal materials, tooth cavity fillers, or dentin.

In some embodiments, an matrix or scaffold (e.g., an acellular matrix or scaffold) is provided. A scaffold can be mammalian tooth-shaped or shaped to fit in a natural or artificial cavity or chamber of a mammalian tooth. A matrix can be suitable for insertion into or partially or fully filling a natural or artificial cavity or chamber of a mammalian tooth. A matrix or scaffold can include bioactive agents (e.g., chemotactic, osteogenic, dentinogenic, amelogenic, or cementogenic agents) described herein (e.g., Wnt3a, BMP-7, VEGF, bFGF, or NGF) or an active analog thereof. A matrix or scaffold can be implanted with or without exogenously applied cells. For example, a matrix or scaffold including Wnt3a, BMP-7, VEGF, bFGF, or NGF, or nucleic acids encoding, such can be introduced onto or into a tooth (e.g., into a tooth chamber) so as to promote odontoblastic differentiation of a progenitor cell, promote cell migration (e.g., progenitor cell migration), regenerate pulp tissue (e.g., vascularized pulp tissue), or regenerate neodentin.

In some embodiments, a composition described herein is used to perform a dental, endodontic or root canal procedure on a mammalian tooth in need thereof. The method can include exposing traumatized or diseased dental pulp tissue in the tooth pulp chamber and/or root canal; and capping or filling at least a portion of the tooth pulp chamber and/or root canal with a composition comprising a bioactive ingredient. The bioactive ingredient can promote angiogenic, odontogenic, fibrogenic, or neurogenic development. In some embodiments, the composition does not include a living cell during the capping or filling. In some embodiments, the method further comprises removing traumatized or diseased dental pulp tissue from the tooth to create a tooth pulp chamber and/or root canal substantially devoid of traumatized or diseased tissue. In some embodiments, the composition comprises a matrix or scaffold.

Bioactive Agents

A composition described herein can include a chemotactic, osteogenic, dentinogenic, amelogenic, or cementogenic compound incorporated into a matrix scaffold. As used herein, a chemotactic compound is a compound that attracts cells. An osteogenic compound is a compound that encourages new bone synthesis. A dentinogenic compound is a compound that encourages new dentin synthesis. An amelogenic compound is a compound that encourages tooth enamel synthesis. A cementogenic compound is a compound that encourages cementum synthesis.

A composition described herein can include any bioactive agent that promotes angiogenic, odontogenic, fibrogenic, or neurogenic development. A composition described herein can include more than one bioactive ingredient, for example two, three, four, or more bioactive ingredients.

A bioactive ingredient can be as described in US Pat Pub No. 2012/0282573, incorporated herein by reference. Non-limiting examples of bioactive agents include platelet-derived growth factor (PDGF), endothelial cell growth factor (ECGF), transforming growth factor-β1 (TGF-β1), epidermal growth factor (EGF), stromal cell-derived factor-1 (SDF1), a bone morphogenetic protein (BMP), a growth and differentiation factor (GDF), insulin-like growth factor-1 (IGF1), vascular endothelial growth factor (VEGF), fibroblast growth factor (FGF), a dentin matrix protein, a dentin sialoprotein, a bone sialoprotein, amelogenin, an integrin, vascular endothelial growth factor (VEGF), basic fibroblast growth factor (bFGF), angiogenin, angiopoietin-1, del-1, follistatin, granulocyte colony-stimulating factor (G-CSF), hepatocyte growth factor/scatter factor (HGF/SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-α (TGF-α), transforming growth factor-β (TGF-β), tumor necrosis factor-α (TNF-α), matrix metalloproteinase (MMP), angiopoietin 1 (ang1), ang2, delta-like ligand 4 (DLL4), connective tissue growth factor (CTGF), bone morphogenic protein (BMP), nerve growth factor (NGF), brain derived nerve factor (BDNF), NT-4, and NT-3.

A bioactive ingredient can be from any mammalian species. In some embodiments, the bioactive ingredient is a human bioactive ingredient, particularly when the mammal being treated is a human. The bioactive ingredient can be a recombinant bioactive ingredient.

A composition described herein can include an antibiotic or an analgesic. A composition described herein can include an antibiotic. Exemplary antibiotics are penicillin V potassium, amoxicillin, augmentin, clindamycin or azithromycin.

A composition described herein can include an analgesic. Exemplary analgesics are paracetamol, diclofenac, ketoprofen, aspirin, naproxen, indomethacin, ketorolac, ibuprofen, piroxicam, celecoxib, meloxicam, mefenemic acid, rofecoxib, nimesulide or a prostaglandin.

Concentrations for a bioactive agent described herein are generally provided as mass per unit volume of solution, where the solution can be introduced into the matrix or scaffold.

A bioactive agent(s) can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Wnt3a

A composition described herein for regeneration of dental pulp or dentin can include a Wnt polypeptide (e.g., Wnt3a). Such a composition can include a Wnt polypeptide in combination with other bioactive agents, such as VEGF, bFGF, BMP-7, or NGF.

Wnt3a can be present in a composition described herein at about 0.1 ng to about 1,000 mg Wnt3a per ml of solution. For example, Wnt3a can be present in a composition described herein at about 0.1 ng to about 100 mg Wnt3a per ml of solution. As another example, Wnt3a can be present in a composition described herein at about 0.1 ng to about 10 mg Wnt3a per ml of solution. As another example, Wnt3a can be present in a composition described herein at about 0.1 ng to about 1 mg Wnt3a per ml of solution. As another example, Wnt3a can be present in a composition described herein at about 0.1 ng to about 0.1 mg Wnt3a per ml of solution. As another example, Wnt3a can be present in a composition described herein at about 0.1 ng to about 100 μg Wnt3a per ml of solution. As another example, Wnt3a can be present in a composition described herein at about 0.1 ng to about 10 μg Wnt3a per ml of solution. As another example, Wnt3a can be present in a composition described herein at about 0.1 ng to about 1 μg Wnt3a per ml of solution. As another example, Wnt3a can be present in a composition described herein at about 0.1 ng to about 100 ng Wnt3a per ml of solution. As another example, Wnt3a can be present in a composition described herein at about 0.1 ng to about 10 ng Wnt3a per ml of solution. As another example, Wnt3a can be present in a composition described herein at about 10 ng Wnt3a per ml of solution.

Solutions containing above described concentrations of Wnt3a can be introduced to or combined with a matrix or scaffold.

As shown herein, canonical Wnt signaling pathway is pivotal for odontoblast specification or differentiation from progenitor cells, such as mesenchymal stem cells, and can orchestrate dental pulp or dentin regeneration as, e.g., a microencapsulated, translatable medical device, which can be free of cell transplantation. Such an approach can replace, for example, root canal materials, tooth cavity fillers, or other dental fillers (see Example 3).

As established herein Wnt3a (wingless-type MMTV integration site family, member 3A) can promote odontoblastic differentiation of a progenitor cell, promote cell migration, regenerate vascularized pulp tissue, or regenerate neodentin.

Wnt protein alone induced dental pulp and dentin regeneration. Wnt protein in combination with, for example, BMP-7 or PDGF can further enhanced dental pulp and dentin regeneration. Wnt protein can augment PDGF and BMP-7 signaling pathways, and can therefore further promote dental pulp or dentin regeneration.

In some embodiments, Wnt3a can be used to promote odontoblastic differentiation of a progenitor cell, e.g. a mesenchyme stem cell, from alveolar bone marrow or periodontal ligament. Wnt3a can also be used to promote cell migration. In vivo delivery of Wnt3a can regenerate vascularized pulp tissue in, e.g., endodontically treated root canals. In vivo delivery of Wnt3a can regenerate neodentin with polarized odontoblasts on native tubular dentin surface, by recruitment of host endogenous cells.

In some embodiments, a Wnt3a polypeptide or a nucleic acid encoding such can be contacted with a tooth, bone, dental pulp, or other dental tissue. As described herein, such contact can promote odontoblastic differentiation of a progenitor cell, promote cell migration (e.g., progenitor cell migration), regenerate pulp tissue (e.g., vascularized pulp tissue), or regenerate neodentin. For example, Wnt3a polypeptide or a nucleic acid encoding such can be introduced onto or into a tooth (e.g., into a tooth chamber) so as to promote odontoblastic differentiation of a progenitor cell, promote cell migration (e.g., progenitor cell migration), regenerate pulp tissue (e.g., vascularized pulp tissue), or regenerate neodentin.

A Wnt3a polypeptide is understood to be encoded by a WNT3A gene (see generally, Saitoh et al. 2001 Biochem Biophys Res Commun 284(5), 1168-1175). The WNT gene family consists of structurally related genes that encode secreted signaling proteins. These proteins have been implicated in oncogenesis and in several developmental processes, including regulation of cell fate and patterning during embryogenesis. This gene is a member of the WNT gene family. It encodes a protein showing 96% amino acid identity to mouse Wnt3A protein, and 84% to human WNT3 protein, another WNT gene product. This gene is clustered with WNT14 gene, another family member, in chromosome 1q42 region.

A Wnt3a polypeptide can be include an amino acid sequence of SEQ ID NO: 1, SEQ ID NO: 2, SEQ ID NO: 3, or SEQ ID NO: 4, or a sequence at least about 80% identical thereto (e.g., at least about 85%, at least about 90%, at least about 95%, at least about 96%, at least about 97%, at least about 98%, or at least about 99%) where the polypeptide retains Wnt3a activity, or a functional fragment thereof.

A Wnt3a polypeptide can be according to the amino acid sequence associated with SwissProtID P27467, incorporated herein by reference. A Wnt3a polypeptide can be according to the amino acid sequence associated with UniParc P56704, incorporated herein by reference. A Wnt3a polypeptide can be according to the amino acid sequence associated with UniParc Q3SY79, incorporated herein by reference. A Wnt3a polypeptide can be according to the amino acid sequence associated with Uniprot accession Q9GTJ9 (Hydra vulgaris), incorporated herein by reference. A Wnt3a polypeptide can be according to the amino acid sequence associated with SwissProtID P27467 (mouse), incorporated herein by reference.

A Wnta polypeptide can be an active fragment of a full length Wnta peptide (e.g., aminoacid residues 250-350 of human Wnt3a).

A Wnt3a polypeptide can have at least about 80% sequence identity to any of the above sequences and retains Wnt3a activity. For example, a Wnta polypeptide can have at least about 80%, at least about 85%, at least about 90%, at least about 95%, at least about 96%, at least about 97%, at least about 98%, or at least about 99% sequence identity to any of the above sequences where the polypeptide retains Wnta activity.

A composition, scaffold or device described herein can include a Wnt3a polypeptide or a nucleic acid encoding a Wnt3a polypeptide. A Wnta nucleic acid can be a nucleic acid that encodes any of the above described Wnta polypeptide sequences. A Wnta nucleic acid can have at least about 80%, at least about 85%, at least about 90%, at least about 95%, at least about 96%, at least about 97%, at least about 98%, or at least about 99% sequence identity to a nucleic acid encoding any of the above sequences such that the encoded polypeptide retains Wnta activity. In embodiments where a nucleic acid encoding a Wnt3a polypeptide, such nucleic acid can be provided in a vector or construct as described herein.

Wnt3a polypeptide is commercially available from a variety of sources (e.g., ABCAM, Cambridge, Mass.).

Wnt3a is know to be proteolytically processed by TIKI1 and TIKI2, which promote oxidation and formation of large disulfide-bond oligomers, leading to inactivation of WNT3A. A composition described herein can include an inhibitor of TIKI1 and TIKI2. In some embodiments, a Wnt3a polypeptide can be a derivative thereof resistant to proteolytic degradation by, for example, TIKI1 or TIKI2.

BMP-7

A composition described herein for regeneration of dental pulp or dentin can include bone morphogenetic protein 7 (BMP-7). Such a composition can include BMP-7 in combination with other bioactive agents, such as Wnt3a, VEGF, bFGF, or NGF.

BMP-7 can be present in a composition described herein at about 0.1 ng to about 1,000 mg BMP-7 per ml of solution. For example, BMP-7 can be present in a composition described herein at about 0.1 ng to about 100 mg BMP-7 per ml of solution. As another example, BMP-7 can be present in a composition described herein at about 0.1 ng to about 10 mg BMP-7 per ml of solution. As another example, BMP-7 can be present in a composition described herein at about 0.1 ng to about 1 mg BMP-7 per ml of solution. As another example, BMP-7 can be present in a composition described herein at about 0.1 ng to about 0.1 mg BMP-7 per ml of solution. As another example, BMP-7 can be present in a composition described herein at about 0.1 ng to about 100 μg BMP-7 per ml of solution. As another example, BMP-7 can be present in a composition described herein at about 0.1 ng to about 10 μg BMP-7 per ml of solution. As another example, BMP-7 can be present in a composition described herein at about 0.1 ng to about 1 μg BMP-7 per ml of solution. As another example, BMP-7 can be present in a composition described herein at about 0.1 ng to about 500 ng BMP-7 per ml of solution. As another example, BMP-7 can be present in a composition described herein at about 0.1 ng to about 200 ng BMP-7 per ml of solution. As another example, BMP-7 can be present in a composition described herein at about 0.1 ng to about 100 ng BMP-7 per ml of solution. As another example, BMP-7 can be present in a composition described herein at about 0.1 ng to about 10 ng BMP-7 per ml of solution. As another example, BMP-7 can be present in a composition described herein at about 200 ng BMP-7 per ml of solution.

Solutions containing above described concentrations of BMP-7 can be introduced to or combined with a matrix or scaffold.

BMP-7 is commercially available.

VEGF

A composition described herein for regeneration of dental pulp or dentin can include vascular endothelial growth factor (VEGF). Such a composition can include VEGF in combination with other bioactive agents, such as Wnt3a, bFGF, BMP-7, or NGF.

VEGF can be present in a composition described herein at about 0.1 ng to about 1,000 mg VEGF per ml of solution. For example, VEGF can be present in a composition described herein at about 0.1 ng to about 100 mg VEGF per ml of solution. As another example, VEGF can be present in a composition described herein at about 0.1 ng to about 10 mg VEGF per ml of solution. As another example, VEGF can be present in a composition described herein at about 0.1 ng to about 1 mg VEGF per ml of solution. As another example, VEGF can be present in a composition described herein at about 0.1 ng to about 0.1 mg VEGF per ml of solution. As another example, VEGF can be present in a composition described herein at about 0.1 ng to about 100 μg VEGF per ml of solution. As another example, VEGF can be present in a composition described herein at about 0.1 ng to about 10 μg VEGF per ml of solution. As another example, VEGF can be present in a composition described herein at about 0.1 ng to about 1 μg VEGF per ml of solution. As another example, VEGF can be present in a composition described herein at about 0.1 ng to about 500 ng VEGF per ml of solution. As another example, VEGF can be present in a composition described herein at about 0.1 ng to about 200 ng VEGF per ml of solution. As another example, VEGF can be present in a composition described herein at about 0.1 ng to about 100 ng VEGF per ml of solution. As another example, VEGF can be present in a composition described herein at about 0.1 ng to about 10 ng VEGF per ml of solution. As another example, VEGF can be present in a composition described herein at about 33 ng VEGF per ml of solution.

Solutions containing above described concentrations of VEGF can be introduced to or combined with a matrix or scaffold.

VEGF is commercially available.

bFGF

A composition described herein for regeneration of dental pulp or dentin can include basic fibroblast growth factor (bFGF). Such a composition can include bFGF in combination with other bioactive agents, such as Wnt3a, VEGF, BMP-7, or NGF.

bFGF can be present in a composition described herein at about 0.1 ng to about 1,000 mg bFGF per ml of solution. For example, bFGF can be present in a composition described herein at about 0.1 ng to about 100 mg bFGF per ml of solution. As another example, bFGF can be present in a composition described herein at about 0.1 ng to about 10 mg bFGF per ml of solution. As another example, bFGF can be present in a composition described herein at about 0.1 ng to about 1 mg bFGF per ml of solution. As another example, bFGF can be present in a composition described herein at about 0.1 ng to about 0.1 mg bFGF per ml of solution. As another example, bFGF can be present in a composition described herein at about 0.1 ng to about 100 μg bFGF per ml of solution. As another example, bFGF can be present in a composition described herein at about 0.1 ng to about 10 μg bFGF per ml of solution. As another example, bFGF can be present in a composition described herein at about 0.1 ng to about 1 μg bFGF per ml of solution. As another example, bFGF can be present in a composition described herein at about 0.1 ng to about 500 ng bFGF per ml of solution. As another example, bFGF can be present in a composition described herein at about 0.1 ng to about 200 ng bFGF per ml of solution. As another example, bFGF can be present in a composition described herein at about 0.1 ng to about 100 ng bFGF per ml of solution. As another example, bFGF can be present in a composition described herein at about 0.1 ng to about 10 ng bFGF per ml of solution. As another example, bFGF can be present in a composition described herein at about 167 ng bFGF per ml of solution.

It has been discovered that VEGF and bFGF in implanted collagen sponges can be effective in restoring viable tissue in a pulp chamber and root canal when added to collagen sponges and inserted into the pulp chamber after a root canal procedure (see e.g., Example 1).

In some embodiments where the composition comprises a VEGF and bFGF, the composition comprises about 0.001 ng to about 10,000 μg VEGF and about 0.001 ng to about 10,000 μg bFGF per ml of solution. In other embodiments, the composition comprises about 0.01 ng to about 1,000 μg VEGF and about 0.02 ng to about 2,000 μg bFGF per ml of solution. In additional embodiments, the composition comprises about 10 ng to about 200 ng VEGF and about 50 ng to about 500 ng bFGF per ml of solution. In further embodiments, the composition comprises about 33 ng VEGF and about 167 ng bFGF per ml of solution.

Solutions containing above described concentrations of bFGF can be introduced to combined with a matrix or scaffold.

bFGF is commercially available.

NGF

A composition described herein for regeneration of dental pulp or dentin can include nerve growth factor (NGF). Such a composition can include NGF in combination with other bioactive agents, such as Wnt3a, VEGF, bFGF, or BMP-7.

NGF can be present in a composition described herein at about 0.1 ng to about 1,000 mg NGF per ml of solution. For example, NGF can be present in a composition described herein at about 0.1 ng to about 100 mg NGF per ml of solution. As another example, NGF can be present in a composition described herein at about 0.1 ng to about 10 mg NGF per ml of solution. As another example, NGF can be present in a composition described herein at about 0.1 ng to about 1 mg NGF per ml of solution. As another example, NGF can be present in a composition described herein at about 0.1 ng to about 0.1 mg NGF per ml of solution. As another example, NGF can be present in a composition described herein at about 0.1 ng to about 100 μg NGF per ml of solution. As another example, NGF can be present in a composition described herein at about 0.1 ng to about 10 μg NGF per ml of solution. As another example, NGF can be present in a composition described herein at about 0.1 ng to about 1 μg NGF per ml of solution. As another example, NGF can be present in a composition described herein at about 0.1 ng to about 500 ng NGF per ml of solution. As another example, NGF can be present in a composition described herein at about 0.1 ng to about 200 ng NGF per ml of solution. As another example, NGF can be present in a composition described herein at about 0.1 ng to about 100 ng NGF per ml of solution. As another example, NGF can be present in a composition described herein at about 0.1 ng to about 10 ng NGF per ml of solution.

NGF can be present in a composition described herein at about 0.2 ng to 500 ng NGF per ml of solution. For example, NGF can be present in a composition described herein at about 0.5 ng to 100 ng NGF per ml of solution. As another example, NGF can be present in a composition described herein at about 1 ng to 10 ng NGF per gram of composition.

In some embodiments where the composition comprises BMP-7 and NGF, the composition comprises about 0.2 ng to 10,000 ng BMP-7 and about 0.2 ng to 500 ng NGF per ml of solution. In other embodiments, the composition comprises about 1 ng to 1000 ng BMP-7 and about 0.5 ng to 100 ng NGF per ml of solution. In additional embodiments, the bioactive ingredient composition comprises about 5 ng to 50 ng BMP-7 and about 1 ng to 10 ng NGF per ml of solution.

Solutions containing above described concentrations of NGF can be introduced to combined with a matrix or scaffold.

NGF is commercially available.

Matrix or Scaffold

One aspect of the present disclosure provides a matrix or scaffold suitable for insertion into a tooth pulp chamber, where a composition including one or more bioactive agents described herein is included in or on the matrix or scaffold. The matrix or scaffold including a composition of bioactive agent(s) can promotes vascular tissue formation or nerve formation into the matrix or scaffold when inserted into the tooth pulp chamber. In some embodiments, the matrix or scaffold does not comprise a living cell. Such materials can be used in a dental, endodontic or root canal procedure.

As used herein, a “matrix” is an amorphous structure, e.g., a gel, in which one or more bioactive ingredients can be suspended. A “scaffold” is understood to have a secondary or tertiary structure (e.g., a columnar structure or a porous structure, such as in a typical collagen sponge, e.g., with fairly uniform pores between about 250 and 400 μM, in which one or more bioactive ingredients can permeate). The present disclosure is not limited to any particular matrix or scaffold. Preferably, the matrix or scaffold is biodegradable.

In some embodiments, the matrix or scaffold includes a hydrogel. A hydrogel is understood to have a network of polymer chains that are hydrophilic, sometimes found as a colloidal gel in which water is the dispersion medium. Hydrogels can be highly absorbent (e.g., over about 80%, 85%, 90%, 95%, 99%, or 99.9% water) natural or synthetic polymers. Hydrogels can also possess a degree of flexibility similar to natural tissue, due to their significant water content. A hydrogel can include, for example, polyvinyl alcohol, sodium polyacrylate, acrylate polymers or copolymers with hydrophilic groups. Natural hydrogels can include agarose, methylcellulose, hyaluronan, or other naturally derived polymers.

A composition including one or more bioactive agents can be combined with the matrix or scaffold by any means known in the art. For example, a composition including one or more bioactive agents can be injected into a matrix or scaffold. As another example, a composition including one or more bioactive agents can be mixed into a matrix or scaffold. As another example, Further, a composition including one or more bioactive agents can be encapsulated in the matrix or scaffold, or chemically tethered to, or absorbed in, the matrix or scaffold, by methods known in the art.

A matrix or scaffold can be placed over a surgically prepared tooth cavity or into a pulp chamber after removing pathological tissues, including decayed enamel and dentin. For matrix or scaffold materials such as alginate hydrogel that are placed into the prepared tooth cavity, blood, tissue, fluid, or water can be absorbed into the biomaterial. The pulp chamber can be sealed or the diseased tooth restored while the matrix or scaffold remains in place or after removal of the material.

In some embodiments, a biomaterial device can be removed after about 24 hours. For example, a biomaterial device can be removed after about 24 hours to about 48 hours. As another example, a biomaterial device can be removed after about 48 hours.

A matrix or scaffold can provide a substrate for the growth of cells or formation of tissue. Useful properties of a matrix or scaffold are porosity, biocompatibility and biodegradability, the ability to support cell growth, or its use as a controlled gene- and protein-delivery vehicle (Murphy 1999). The three-dimensional macromolecular structure provided by a scaffold can guide the final shape of bioengineered tissues (Murphy 1999).

A scaffold described herein can have the shape of any mammalian tooth or cavity or chamber therein. For example, a scaffold can have the shape of a human incisor, a human cuspid, a human bicuspid or a human molar, or a cavity or chamber therein. A matrix described herein can conform to a natural or artificial cavity or chamber of any mammalian tooth.

Many studies have investigated the role and action of exogenous growth factors in a carrier to deliver the growth factor to an implantation site. Although the carrier may not contribute any additional factors necessary for tissue formation, it can still be an important component of the growth process (Wozney 1990). One of the carrier functions is to maintain the factor at the site of implantation and thus enhance its local concentration. The carrier also serves as an environment in which tissue can form and therefore helps to define the region in which new tissue can be formed (Whang 1998). Collagenous or synthetic carriers have been used as delivery vehicles, and their physicochemical properties, together with the microenvironment they create, play a role in the inductive outcome. Carriers can be solid xenogenic (e.g., hydroxyapatite) (Kuboki 1995, Murata 1998), solid alloplastic (polyethylene polymers) materials (Saito 1998, Isobe 1999), or gels of autogenous (Sweeney 1995, Schwartz 1998), allogenic (Bax 1999, Viljanen 1997), or alloplastic origin (Santos 1998), and combinations of the above (Alpaslan 1996).

A matrix or scaffold can further comprise any other bioactive molecule, for example an antibiotic or a chemotactic growth factor. In some embodiments, the matrix or scaffold is strengthened, through the addition of, e.g., human serum albumin (HSA), hydroxyethyl starch, dextran, or combinations thereof. Suitable concentrations of these compounds for use in the compositions of the application are known to those of skill in the art, or can be readily ascertained without undue experimentation.

The concentration of a compound in a matrix or scaffold will vary with the nature of the compound, its physiological role, and desired therapeutic or diagnostic effect. A therapeutically effective amount is generally a sufficient concentration of therapeutic agent to display the desired effect without undue toxicity.

A compound can be incorporated into a matrix or scaffold by any known method. In some embodiments, the compound is imbedded in a gel, e.g., a collagen gel incorporated into the pores of a matrix or scaffold scaffold, as described in the Example.

Alternatively, chemical modification methods may be used to covalently link the compound on the surface of a matrix or scaffold. The surface functional groups of a matrix or scaffold can be coupled with reactive functional groups of the compound to form covalent bonds using coupling agents well known in the art such as aldehyde compounds, carbodiimides, and the like. Additionally, a spacer molecule can be used to gap the surface reactive groups and the reactive groups of the biomolecules to allow more flexibility of such molecules on the surface of the matrix. Other similar methods of attaching biomolecules to the interior or exterior of a matrix will be known to one of skill in the art.

A compound can alternatively be introduced into or onto the matrix via a carrier based system, such as an encapsulation vehicle. Such vehicles are useful as slow release compositions. For example, a compound can be micro-encapsulated to provide for enhanced stability or prolonged delivery. Encapsulation vehicles include, but are not limited to, microparticles, liposomes, microspheres, or the like, or a combination of any of the above to provide the desired release profile in varying proportions. Other methods of controlled-release delivery of agents will be known to the skilled artisan. Moreover, these and other systems can be combined or modified to optimize the integration/release of agents within the matrix.

Polymeric microspheres can be produced using naturally occurring or synthetic polymers and are particulate systems in the size range of 0.1 to 500 μm. Polymeric micelles and polymeromes are polymeric delivery vehicles with similar characteristics to microspheres and can also facilitate encapsulation and matrix integration of a compound described herein. Fabrication, encapsulation, and stabilization of microspheres for a variety of payloads are within the skill of the art (see e.g., Varde & Pack (2004) Expert Opin. Biol. 4(1) 35-51). The release rate of the microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and/or oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme).

Liposomes can also be used to integrate a compound with a matrix or scaffold. The agent carrying capacity and release rate of liposomes can depend on the lipid composition, size, charge, drug/lipid ratio, and method of delivery. Conventional liposomes are composed of neutral or anionic lipids (natural or synthetic). Commonly used lipids are lecithins such as phosphatidylcholines, phosphatidylethanolamines, sphingomyelins, phosphatidylserines, phosphatidylglycerols, and phosphatidylinositols. Liposome encapsulation methods are commonly known in the arts (Galovic et al. (2002) Eur. J. Pharm. Sci. 15, 441-448; Wagner et al. (2002) J. Liposome Res. 12, 259-270). Targeted liposomes and reactive liposomes can also be used in combination with the agents and matrix. Targeted liposomes have targeting ligands, such as monoclonal antibodies or lectins, attached to their surface, allowing interaction with specific receptors and/or cell types. Reactive or polymorphic liposomes include a wide range of liposomes, the common property of which is their tendency to change their phase and structure upon a particular interaction (e.g., pH-sensitive liposomes). See, e.g., Lasic (1997) Liposomes in Gene Delivery, CRC Press, Fla.).

A matrix or scaffold can be fabricated with any material recognized as useful by a skilled artisan. Suitable matrix or scaffold materials are discussed in, for example, Ma and Elisseeff, ed. (2005) Scaffolding in Tissue Engineering, CRC, ISBN 1574445219; Saltzman (2004) Tissue Engineering: Engineering Principles for the Design of Replacement Organs and Tissues, Oxford ISBN 019514130X. Non-limiting examples of potentially useful materials for all or part of a matrix or scaffold include poly(ethylene) glycol, poly(lactide), poly(glycolic acid), poly(lactide-co-glycolide), poly(caprolactone), polyanhydride, polyglactin, polycarbonates, polyamides, polyanhydrides, polyamino acids, polyorthoesters, polyacetals, polycyanoacrylates), polyphosphazene, degradable polyurethanes, polyacrylates, ethylene-vinyl acetate polymers and other acyl substituted cellulose acetates and derivatives thereof, polyurethanes, polystyrenes, polyvinyl chloride, polyvinyl fluoride, polyvinyl pyrrolidone, poly(vinylimidazole), chlorosulphonated polyolifins, polyethylene oxide, polyvinyl alcohol, Teflon®, nylon, agarose, alginate (e.g., calcium alginate gel), fibrin, fibrinogen, fibronectin, collagen (e.g., a collagen gel), gelatin, hyaluronic acid, chitin, and other suitable polymers and biopolymers, or analogs, mixtures, combinations, and derivatives of the above.

In some embodiments, the matrix or scaffold comprises a natural polymer. Exemplary natural polymers are collagens, chitosan, and polysaccharides. In other embodiments, a matrix or scaffold comprises a synthetic polymer. Exemplary synthetic polymers are aliphatic polyesters of poly(α-hydroxy acid)s, and polyethylene glycols. Additional synthetic polymers are polylactic acid (PLA), polyglycolic acid (PGA), and mixtures of PLA and PGA (PLGA). In some embodiments, the synthetic polymer is PLGA comprising about 50% PLA and 50% PGA. In other embodiments, a matrix or scaffold comprises a collagen sponge or PLGA.

In some embodiments, a matrix or scaffold is fabricated from a composition that comprises an osteoconductive material. A non-limiting example of an osteoconductive material is hydroxyapatite (HA). HA has been used as a bone substitute for many years because of its excellent biocompatibility and high bioactivity (Liao 2006, Nebahat 2006, Lijun 2006, Wei 2003).

Although HA has good bioactivity and osteoconductivity, it is very brittle and has poor inherent tensile properties. Therefore, in some embodiments, the HA is combined with ε-polycaprolactone (PCL). PCL is a good bone matrix or scaffold material because it takes several years to degrade in vivo and is biocompatible, relatively inexpensive, and available in large quantities (Rich 2002, Kim 2004). The combination of PCL and HA (PCL-HA) provides a desirable combination of bioactivity, biodegradability, and strength (Patcharaporn 2005, Rezwan 2006, Landis 1995, Ziv 1994). The material of composite PCL-HA has been deemed to possesses the optimal matrix or scaffold properties of biocompatibility, cell-adhesion, proliferation, and differentiation (Zhao 2008). In some embodiments, a matrix or scaffold comprises a mixture of about 80 wt % polycaprolactone and about 20 wt % hydroxyapatite. In other embodiments, a matrix or scaffold comprises anywhere from about 60 wt % polycaprolactone and about 40 wt % hydroxyapatite to about 95 wt % polycaprolactone and about 5 wt % hydroxyapatite. For example, a matrix or scaffold can comprise about 70 wt % polycaprolactone and about 30 wt % hydroxyapatite. As another example, a matrix or scaffold can comprise about 90 wt % polycaprolactone and about 10 wt % hydroxyapatite.

In some embodiments, a matrix or scaffold has a high porosity. Such a porous structure provides space for cell migration, adhesion, and the ingrowth of new bone tissue (Gazdag 1995, Rezwan 2006, Mano 2004, Shin 2003, Kim 2001, Leong 2003).

Pores and channels of a scaffold can be engineered to be of various diameters. For example, the pores of the scaffold can have a diameter range from micrometers to millimeters. In some embodiments, the pores of the matrix material include microchannels. Microchannels can have an average diameter of about 0.1 μm to about 1,000 μm, e.g., about 50 μm to about 500 μm (for example about 100 μm, 150 μm, about 200 μm, about 250 μm, about 300 μm, about 350 μm, about 400 μm, about 450 μm, about 500 μm, or about 550 μm). One skilled in the art will understand that the distribution of microchannel diameters can have any distribution including a normal distribution or a non-normal distribution. In some embodiments, microchannels are a naturally occurring feature of the matrix material(s). In other embodiments, microchannels are engineered to occur in the matrix materials.

Methods of replacing a tooth in the mouth of a mammal is also provided herein. In these embodiments, the tooth is absent and a tooth socket is present in the mouth at the position of the absent tooth. The methods comprise implanting an acellular scaffold having the shape of the missing tooth into the tooth socket.

Several methods are used for fabrication of porous scaffolds, including particulate leaching, gas foaming, electrospinning, freeze drying, foaming of ceramic from slurry, and the formation of polymeric sponge (Mikos 1994, Mooney 1996, Qing 2002, Sylvain 2006). However, scaffolds prepared by using these methods have some shortcomings in controlling the structure and interconnectivity of pores, which may limit their application in terms of cell penetration in tissue engineering (Yeong 2004, Tan 2003).

In some embodiments, the methods further comprise making a model of the absent tooth by computer aided design (CAD) and synthesizing the scaffold with a bioplotter. Such methods can provide scaffolds with high porosity and good interconnectivity. Rapid prototyping (RP) methods such as fused deposition modeling, selective laser sintering, 3D printing, multiphase jet solidification, and 3D plotting have been proposed (Hutmacher 2001, Moroni 2006).

A key feature of rapid prototyping is the solid freeform fabrication (SFF) process: 3D computer models are cut into sequences of layers which are used to construct complex objects layer-by-layer. The layers are produced via solidification of melts, layer photopolymerization or bonding of particles using either laser beam induced sintering (selective laser sintering) or special binders (Landers 2002). Recently, a specialized rapid prototyping system (Bioplotte™, EnvisionTec, Germany) has been introduced, enabling the design and fabrication of anatomically shaped scaffolds with varying internal architectures, thereby allowing precise control over pore size, porosity, permeability, and stiffness (Landers 2002; Landers 2005). The prototyping process using the Bioplotter™ for fabricating a tissue-specific PCL-HA scaffold requires 3D morphological information of the target tissue or tissue defect, which can be obtained by computer tomography (CT) or magnetic resonance imaging (MRI). When an absent tooth has a counterpart on the other side of the mouth, that counterpart can be used as a model to design the scaffold for the missing tooth.

Information obtained above is then used to design a functional scaffold by CAD and is transferred to the Bioplotter™ system. In that system, the Bioplotter™ machine melts and dispenses the scaffold material (e.g., PCL-HA) in layer-by-layer on a collecting plate. Pores, for example microchannels, can be created as part of the design. The fabricated 3D scaffolds through the RP system result in significant cell penetration, and thus possess the properties of ideal scaffolds (Heo 2007). These 3D scaffolds may have potential for clinical application by providing patient tissue-specific anatomical shape as well as an optimized internal microstructure for the nutrient transportation and vascularization. Further details of these methods are provided in PCT Publication WO2009/006558, incorporated by reference.

A method of making a tooth scaffold is additionally provided. The method comprises synthesizing an acellular scaffold in the shape of a mammalian tooth and adding a compound described herein. In some embodiments of these methods, the tooth is shaped like a tooth that is absent in a mammal, and the method further comprises making a model of an absent tooth by computer aided design (CAD), and synthesizing the scaffold with a bioplotter. Where the absent tooth has a counterpart in the mouth, e.g., a molar, the method further comprises making a CT scan of the analogous molar, for example on the other side of the mouth, where the CAD utilizes CT scan data of the second molar to design the scaffold.

In some embodiments of these methods, a bioactive agent described herein, or nucleic acid encoding such, is included in the scaffold.

In some embodiments of these methods, compounds included in the scaffold can be Wnt3a polypeptide, platelet-derived growth factor (PDGF), endothelial cell growth factor (ECGF), transforming growth factor-β1 (TGF-β1), epidermal growth factor (EGF), hepatocyte growth factor (HGF), stromal cell-derived factor-1 (SDF1), a bone morphogenetic protein (BMP), a TGF-β, a growth and differentiation factor (GDF), insulin-like growth factor-1 (IGF1), vascular endothelial growth factor (VEGF), fibroblast growth factor (FGF), a dentin matrix protein, a dentin sialoprotein, a bone sialoprotein, amelogenin, or an integrin.

In other embodiments, the scaffold is fabricated from a composition that comprises an osteoconductive material. As discussed above, an example of a useful osteoconductive material is hydroxyapatite. A further example is a mixture of ε-polycaprolactone and hydroxyapatite as discussed above. In various embodiments of these methods, the scaffold comprises microchannels having a diameter of between 50 and 500 μm. In additional embodiments, the scaffold further comprises a nonporous cap.

Progenitor Cell

As used herein, a progenitor cell (e.g., a stem cell) is a relatively undifferentiated cell capable of self-renewal through mitotic cell division and also capable of differentiating into more specialized cell types. As is known in the art, stem cells include embryonic stem cells, which are totipotent, i.e., capable of differentiating into all cell types of the organism from which they were derived, and adult stem cells, which are pluripotent (capable of differentiating into almost all cell types including types from all three germ layers), multipotent (capable of differentiating into several cell types of a closely related family of cells), or unipotent (capable of differentiating into only one type of cell but distinguished from non-stem cells by the ability to self-renew by mitosis).

As used herein, a dental stem cell (DSC; also known as tooth-derived stem cell, TSC) is a progenitor cell derived from vertebrate tooth pulp. A DSC can be from any tooth of any vertebrate that has teeth. In some embodiments, the dental stem cell is derived from a deciduous tooth. In other embodiments, the dental stem cell is derived from a premolar, a molar, an incisor or a canine. DSCs are capable of differentiating into cells of all three germ types, and thus are pluripotent cells.

Dental Tissue

As described herein, dental tissue can be regenerated upon contact with a Wnt3a polypeptide. A dental tissue can be dental pulp, coronal pulp (e.g., occlusal, mesial, distal, buccal, lingual or floor), radicular pulp, periapical tissue, periapical connective tissue, periodontal tissue, accessory canals, apical foramen, foramina, Zone of Rinaggio, zone of Weil, odontoblastic layer, bone, gum, blood vessels, nerves, nerve plexus of Raschkow, cementum, dentin, sclerotic dentin, tertiary dentin, reactionary dentin, reparative dentin, dentinal tubules, neodentin, or tissues containing any of ameloblasts, fibroblasts, odontoblasts, histiocytes, macrophage, granulocytes, mast cells or plasma cells.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject. Such formulations can be incorporated into or onto a matrix or scaffold.

Individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of regenerating a dental tissue in a subject in need administration of a therapeutically effective amount of a matrix or scaffold including Wnt3a, BMP-7, VEGF, bFGF, or NGF, or combinations thereof, so as to promote odontoblastic differentiation of a progenitor cell, promote cell migration, regenerate vascularized pulp tissue, or regenerate neodentin.

Also provided is a process of regenerating a dental tissue in a subject in need administration of a therapeutically effective amount of Wnt3a polypeptide, so as to promote odontoblastic differentiation of a progenitor cell, promote cell migration, regenerate vascularized pulp tissue, or regenerate neodentin.

As used herein, a dental procedure can be any procedure involving teeth. Exemplary dental procedures include endodontic procedures, which involve tooth pulp. A root canal is a dental procedure where the entire tooth pulp and root canal tissue is removed and replaced with an inert material or a composition described herein that can restore living tissue in the pulp chamber.

One aspect of the present disclosure provides a method of performing a dental, endodontic or root canal procedure on a mammalian tooth in need thereof. The method can include exposing traumatized or diseased dental pulp tissue in the tooth pulp chamber or root canal; and capping or filling at least a portion of the tooth pulp chamber or root canal with a composition comprising a bioactive ingredient (e.g., a scaffold including a composition of bioactive agent(s)). As described herein, the composition can promote angiogenic, odontogenic, fibrogenic, or neurogenic development. In some embodiments, the composition does not comprise a living cell during the capping or filling. In some embodiments, the method further includes removing traumatized or diseased dental pulp tissue from the tooth to create a tooth pulp chamber or root canal substantially devoid of traumatized or diseased tissue. In some embodiments, a matrix or scaffold contains the composition of bioactive agent(s), and the matrix or scaffold is inserted into the tooth chamber or root canal.

One application of the instant disclosure is a root canal procedure, where all pulp tissue is removed from the tooth. A matrix or scaffold described herein can partially or completely replace current endodontic filing materials such as gutta-percha in those methods. The current methods do not exclude the combined use of the matrix or scaffold and current materials such as gutta-percha. Thus, in some embodiments, an inert material is also inserted into the pulp chamber, for example gutta-percha.

The replaced pulp could be due to any condition that a dental, endodontic or root canal procedure is prescribed to remedy. For example, the pulp tissue could have been infected with bacteria. Alternatively, the pulp tissue could have been damaged due to trauma, or there could be a defect in the pulp tissue.

Methods described herein are generally performed on a subject in need thereof. A subject in need of the therapeutic methods described herein can be a subject having, diagnosed with, suspected of having, or at risk for developing an injury, disease or condition associated with dental tissue, such as dental trauma, pulpitis or dental caries. For example, a subject in need of the therapeutic methods described herein can be a subject having, diagnosed with, suspected of having, or at risk for developing reversible pulpitis or other diseases or conditions that conventionally require removal of healthy dental pulp along with diseased pulp. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a matrix or scaffold including Wnt3a, BMP-7, VEGF, bFGF, or NGF, or combinations thereof, is, for example, that amount that would cause the desired therapeutic effect in a subject while minimizing undesired side effects. In various embodiments, an effective amount of a matrix or scaffold including Wnt3a, BMP-7, VEGF, bFGF, or NGF, or combinations thereof, promote odontoblastic differentiation of a progenitor cell, promote cell migration, regenerate vascularized pulp tissue, or regenerate neodentin.

Generally, a safe and effective amount of Wnt3a polypeptide is, for example, that amount that would cause the desired therapeutic effect in a subject while minimizing undesired side effects. In various embodiments, an effective amount of Wnt3a polypeptide promote odontoblastic differentiation of a progenitor cell, promote cell migration, regenerate vascularized pulp tissue, or regenerate neodentin.

When used in the treatments described herein, a therapeutically effective amount of Wnt3a, BMP-7, VEGF, bFGF, or NGF can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to promote odontoblastic differentiation of a progenitor cell, promote cell migration, regenerate vascularized pulp tissue, or regenerate neodentin.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a composition described herein can occur as a single event or over a time course of treatment. For example, a composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for an injury, disease, or condition associated with dental tissue.

A composition described herein can be administered simultaneously or sequentially with another agent, such as an antibiotic, an antiinflammatory, or another agent. For example, a matrix or scaffold including Wnt3a, BMP-7, VEGF, bFGF, or NGF can be administered simultaneously with another agent, such as an antibiotic or an antiinflammatory, or such other agent(s) can be included in or on the matrix or scaffold. Simultaneous administration can occur through administration of separate compositions, each containing one or more of the matrix or scaffold, Wnt3a, BMP-7, VEGF, bFGF, NGF, an antibiotic, an antiinflammatory, or another agent as described above. A matrix or scaffold including Wnt3a, BMP-7, VEGF, bFGF, or NGF can be administered sequentially with an antibiotic, an antiinflammatory, or another agent. For example, a matrix or scaffold including Wnt3a, BMP-7, VEGF, bFGF, or NGF can be administered before or after administration of an antibiotic, an antiinflammatory, or another agent.

The above discussion recites a Wnt3a polypeptide. One of ordinary skill will understand the above discussion can apply equally to a nucleic acid encoding a Wnt3a polypeptide.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (sRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, Mo.; Invitrogen). Several sRNA molecule design programs using a variety of algorithms are known to the art (see e.g., Cenix algorithm, Ambion; BLOCK-iT™ RNAi Designer, Invitrogen; sRNA Whitehead Institute Design Tools, Bioinoformatics & Research Computing). Traits influential in defining optimal sRNA sequences include G/C content at the termini of the siRNAs, Tm of specific internal domains of the sRNA, sRNA length, position of the target sequence within the CDS (coding region), and nucleotide content of the 3′ overhangs.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a matrix or scaffold and a Wnt3a polypeptide or polynucleic acid, BMP-7, VEGF, bFGF, or NGF, an antibiotic, an antiinflammatory, or another agent as described above. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Each of the following publications is incorporated herein by reference in its entirety: US Pat Pub No. 2011/0171607, BIOPULP; US Pat Pub No. 2013/0022989, DENTAL STEM CELL PROGRAMMING; US Pat Pub No. 2011/0236977, DENTAL STEM CELL DIFFERENTIATION; US Pat Pub No. 2012/0282573, TOOTH SCAFFOLDS; US Pat Pub No. 20110171607, BIOPULP; WO 2012/045097, PRODUCTION OF DENTIN, CEMENTUM AND ENAMEL BY CELLS.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

Extracted human incisors were subjected to a root canal treatment. A collagen sponge, with or without bFGF and/or VEGF, was then implanted into the root canal. The incisors were then implanted subcutaneously in immunodeficient mice. The teeth were removed after two weeks and assessed for vascularization in the pulp chamber and root canal.

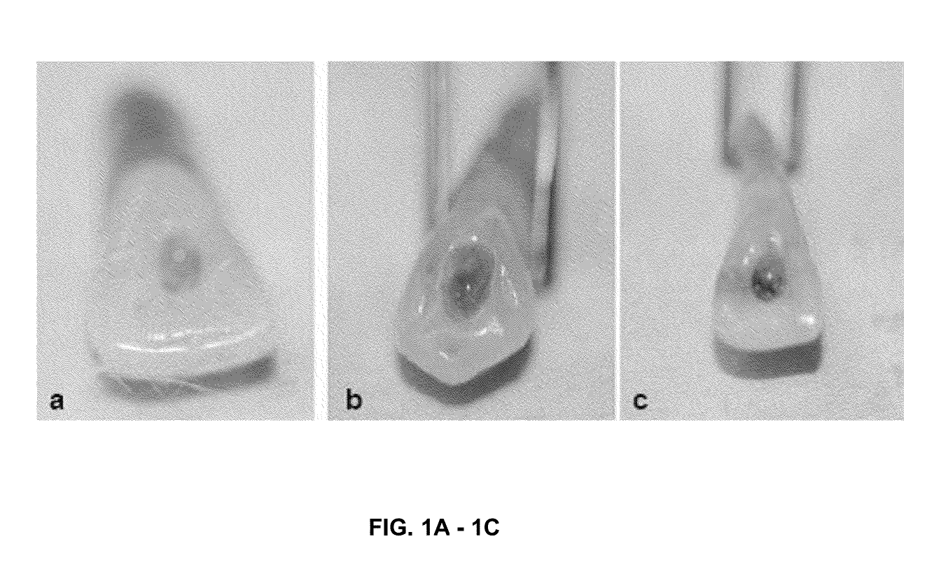

On visual inspection, the teeth treated with a collagen sponge without any bioactive ingredient had no apparent vascular development (FIG. 1a). However, teeth treated with a collagen sponge having bFGF or the combination of bFGF and VEGF showed vascularization in the collagen sponge inserted into the root canal (FIGS. 1b and 1c).

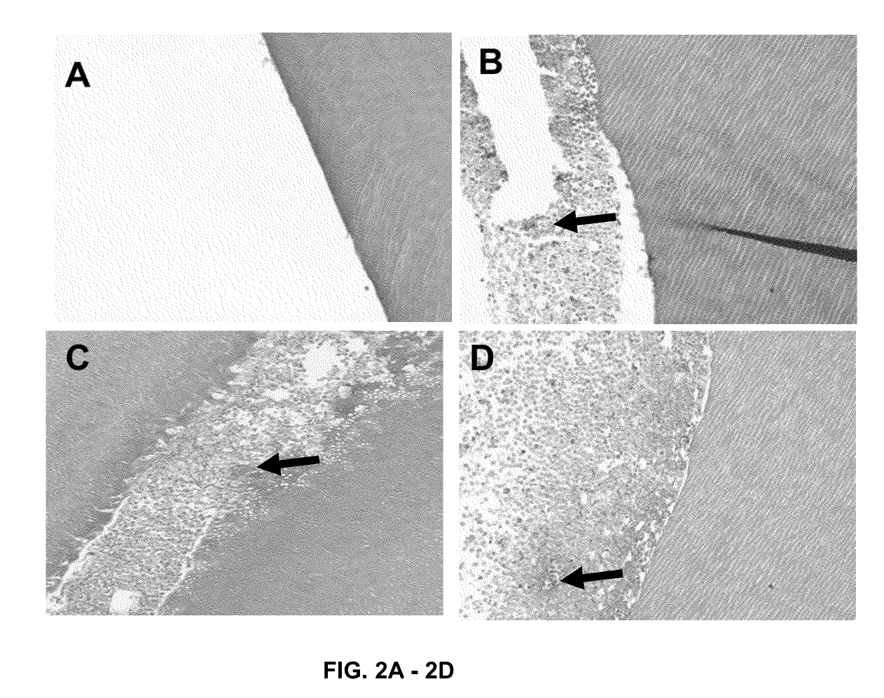

The root canal of the above-treated teeth were further evaluated microscopically. Teeth treated with a collagen sponge without any bioactive ingredient showed no tissue growth in the root canal (FIG. 2A) whereas teeth treated with a collagen sponge with either bFGF or VEGF or the combination of bFGF+VEGF showed vascularization and host tissue ingrowth (FIG. 2B-D). The infiltrating host tissue in those treatments was attached to the dentin.

### Example 2

To following example demonstrates encapsulation and controlled release of an odontogenic bioactive ingredient, bone morphogenic protein-7 (BMP-7), and a neurogenic bioactive ingredient, nerve bioactive ingredient (NGF), in a biocompatible microsphere, poly-d-l-lactic-co-glycolic acid (PLGA). PLGA was fabricated from 50:50 (PLA:PGA), and degraded slowly. BMP-7 and NGF were released gradually upon the degradation of PLGA microspheres over time. After four to six weeks, the release profiles of BMP-7 and NGF were determined by ELISA, and confirmed cumulative release concentration curves. These findings provide the proof of concept for applying BMP-7 and NGF in biocompatible microspheres for the regeneration of dental pulp in vivo.

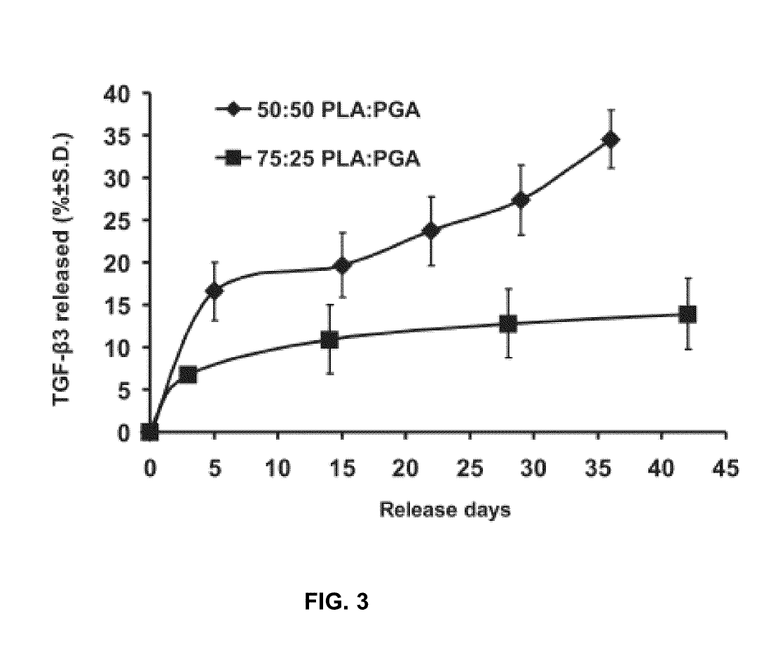

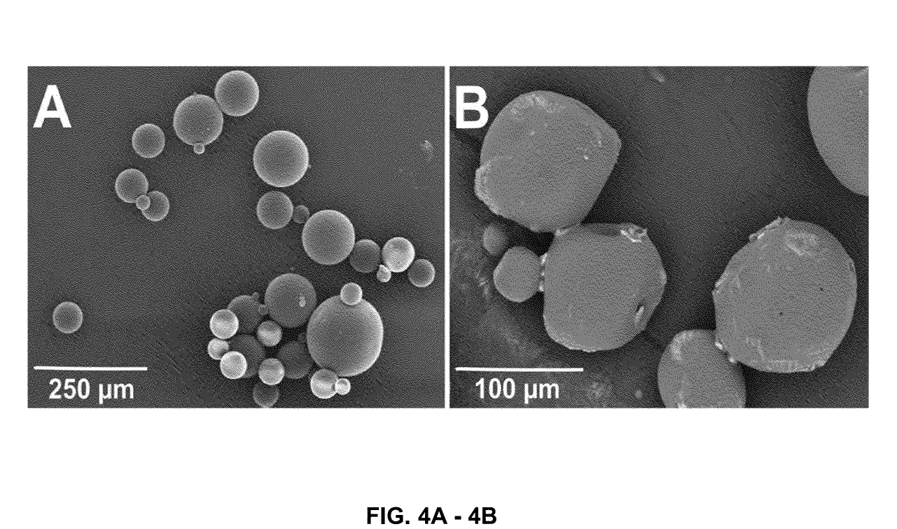

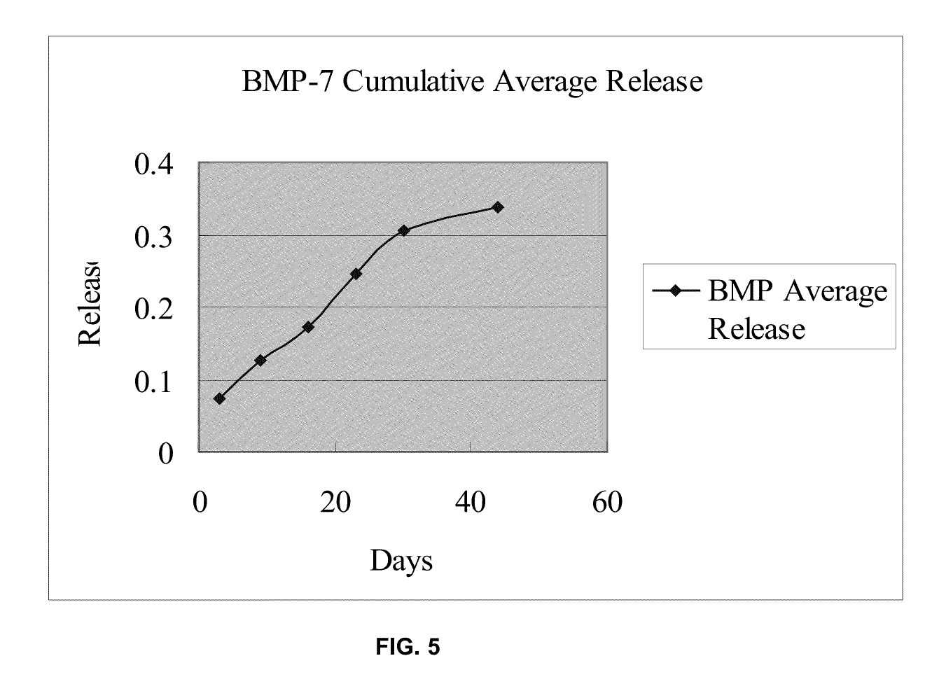

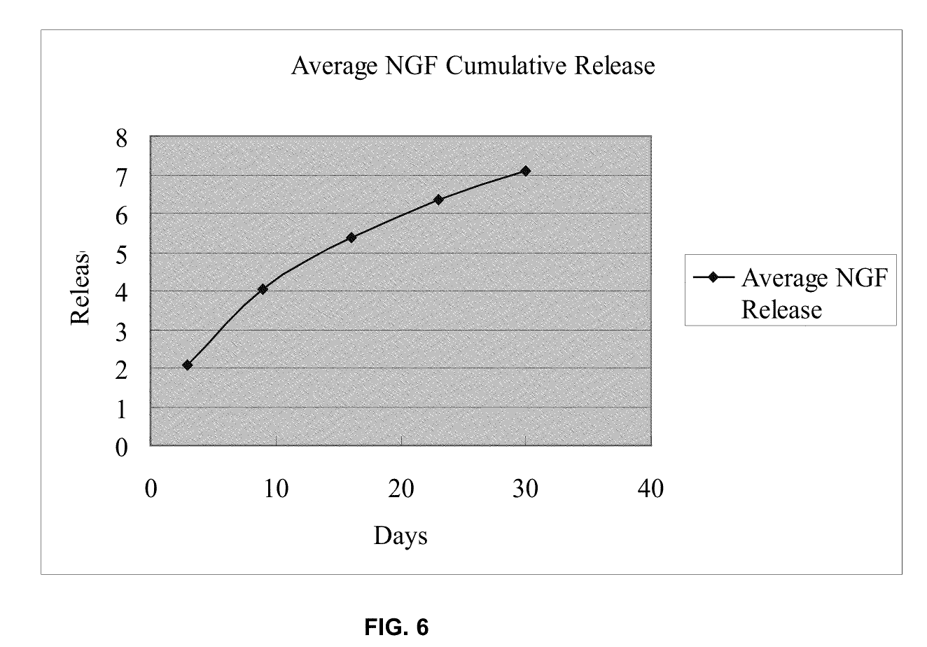

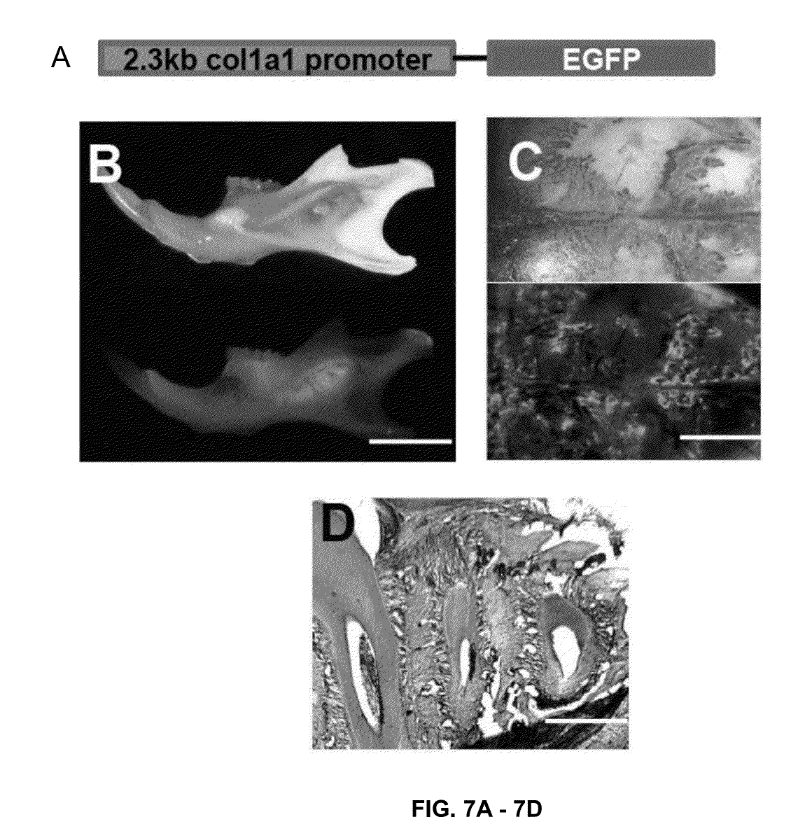

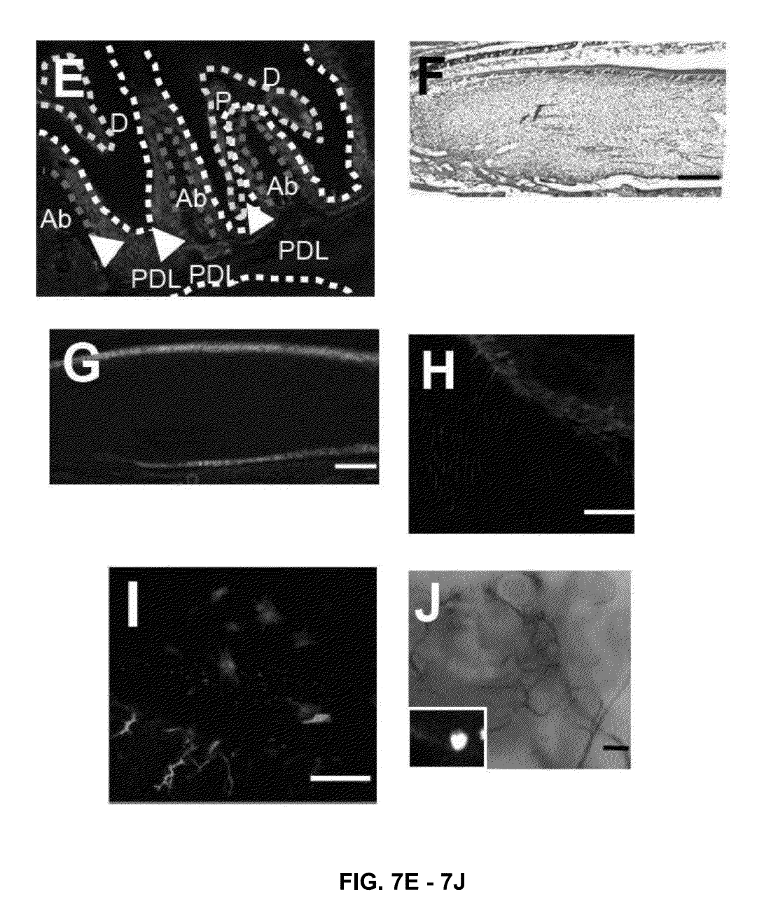

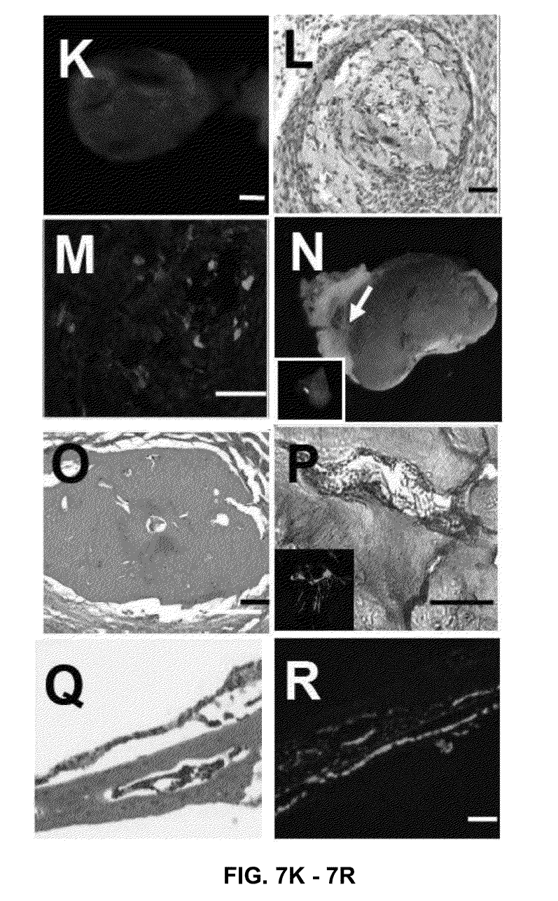

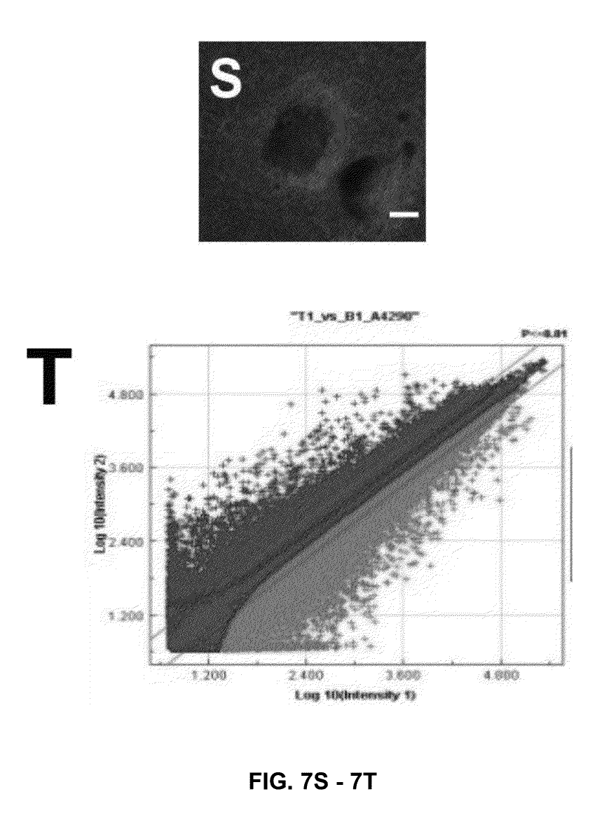

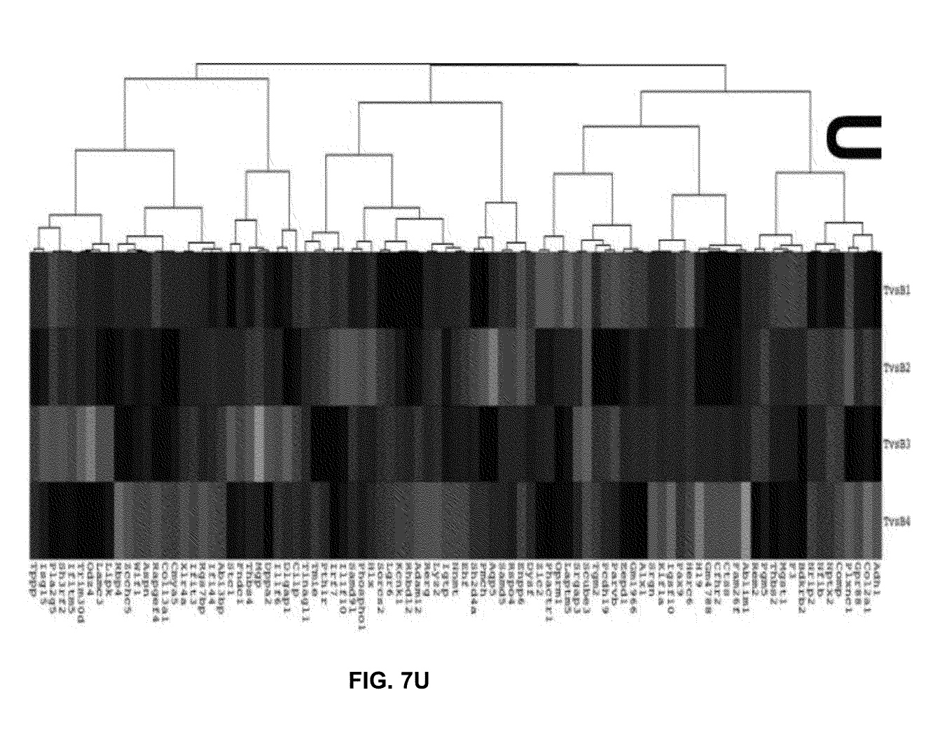

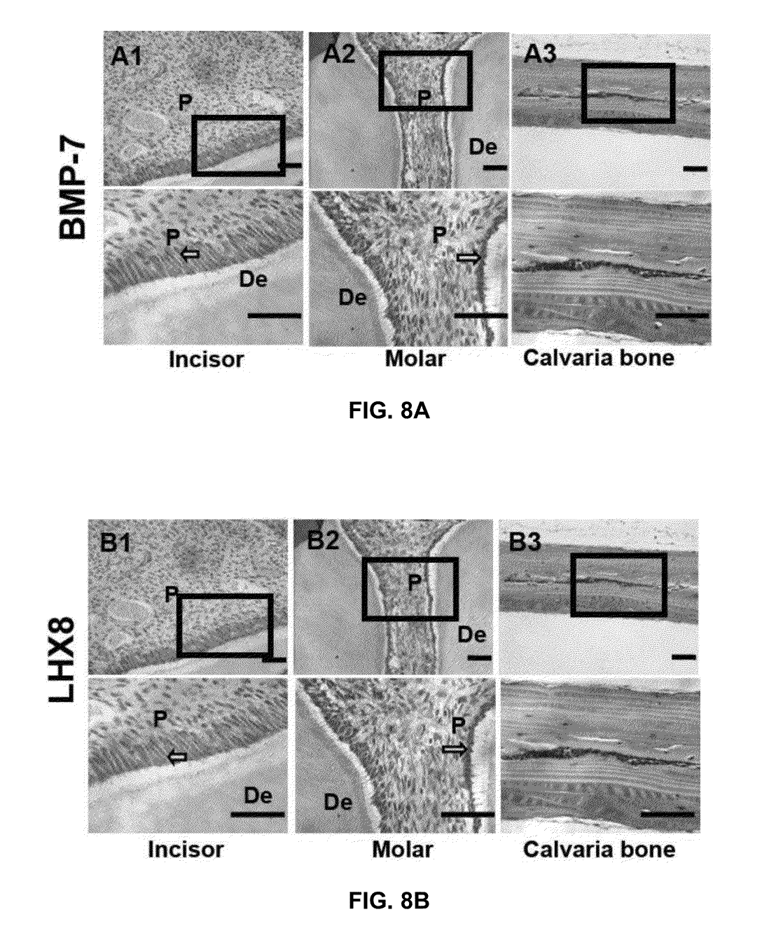

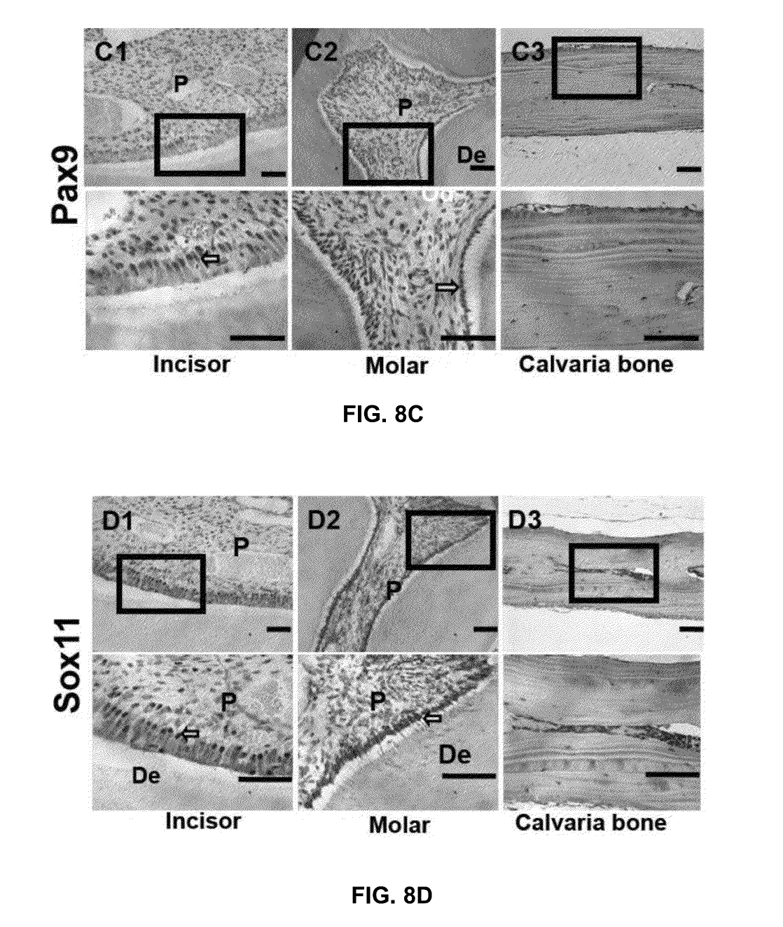

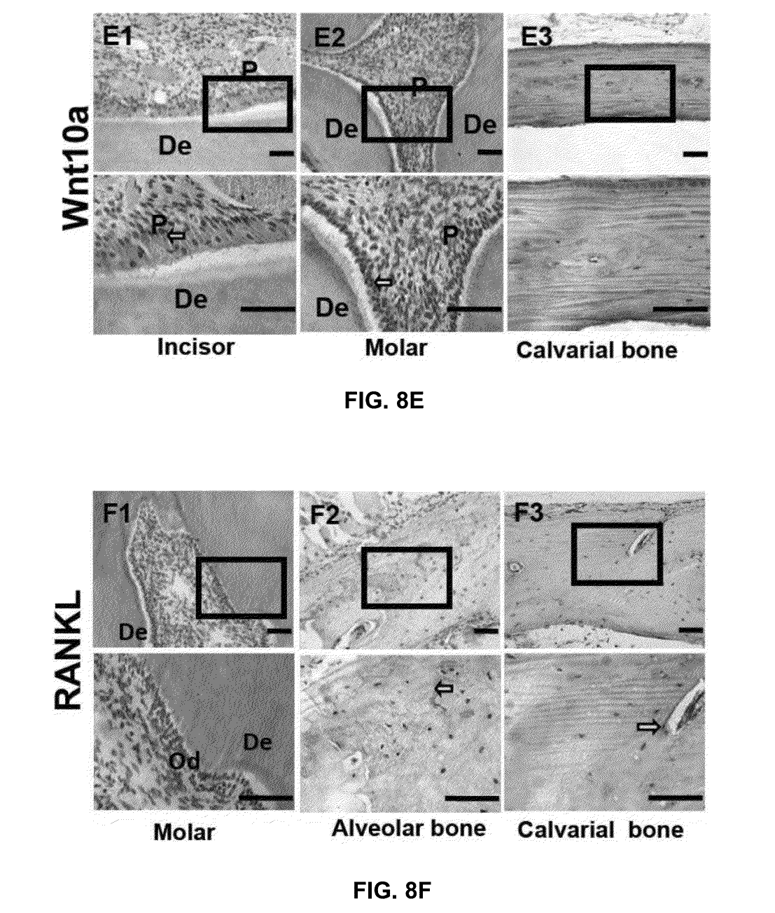

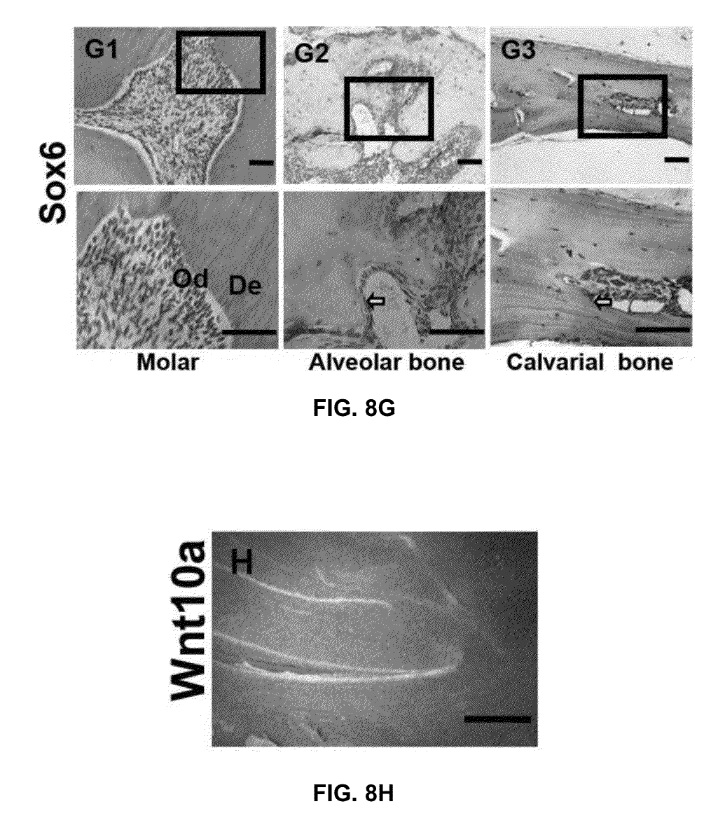

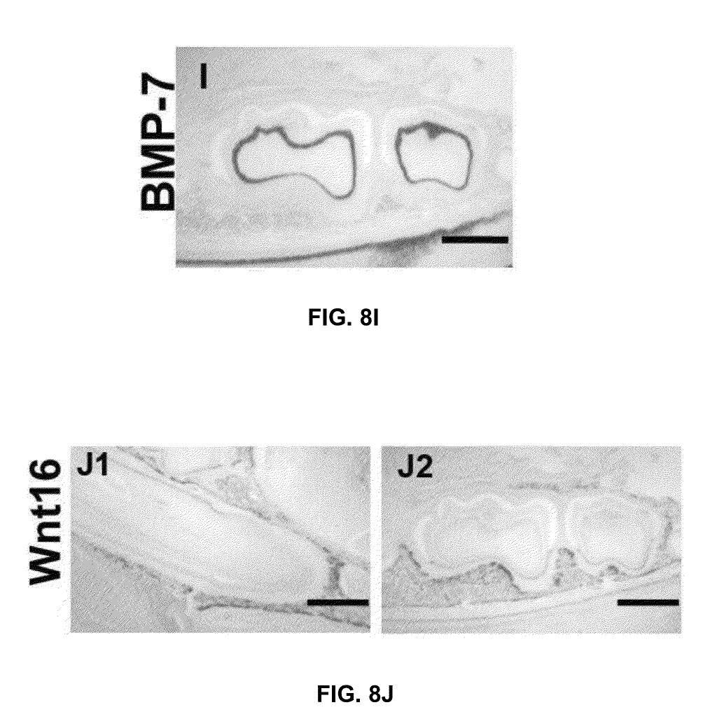

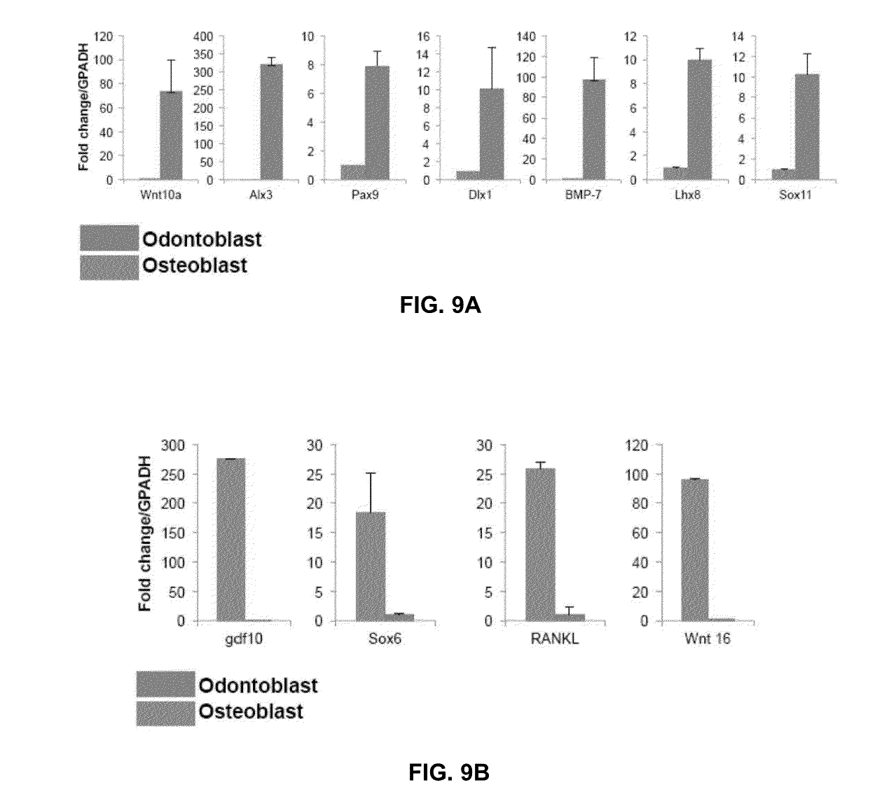

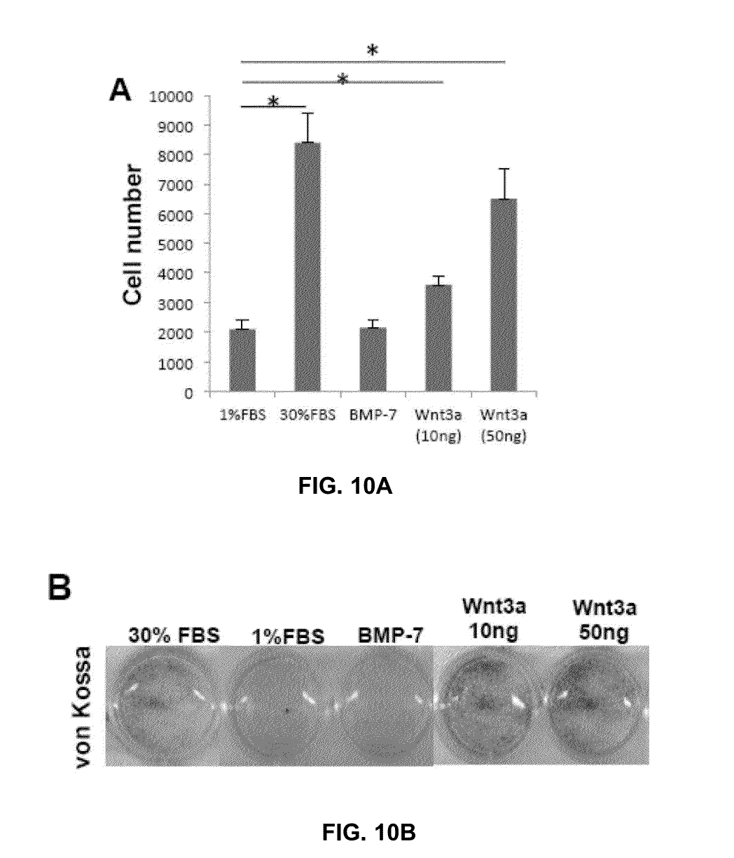

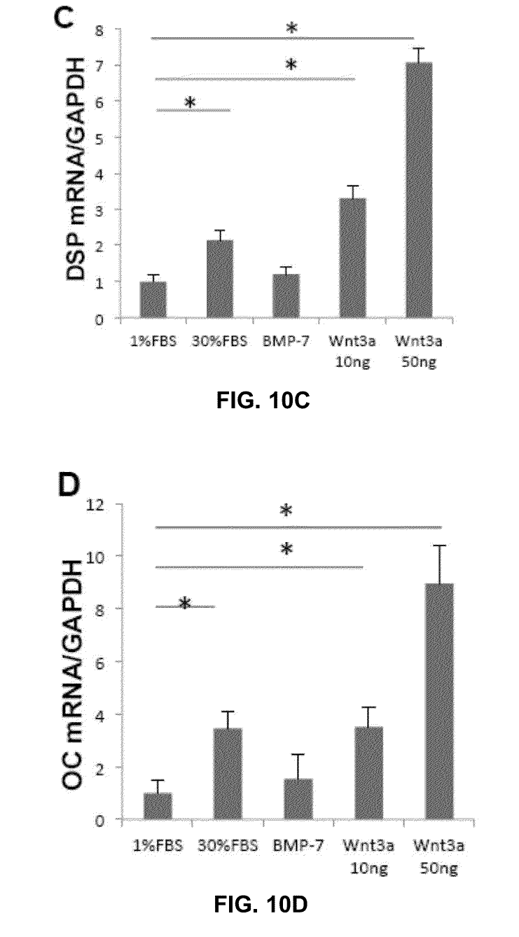

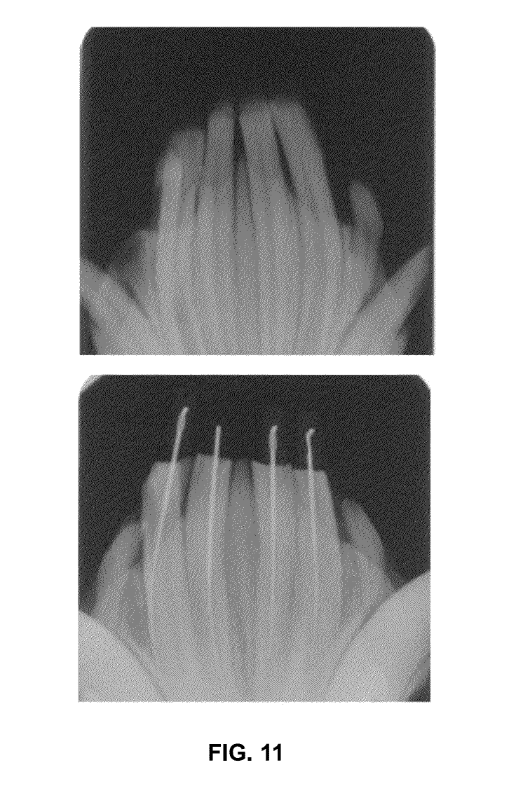

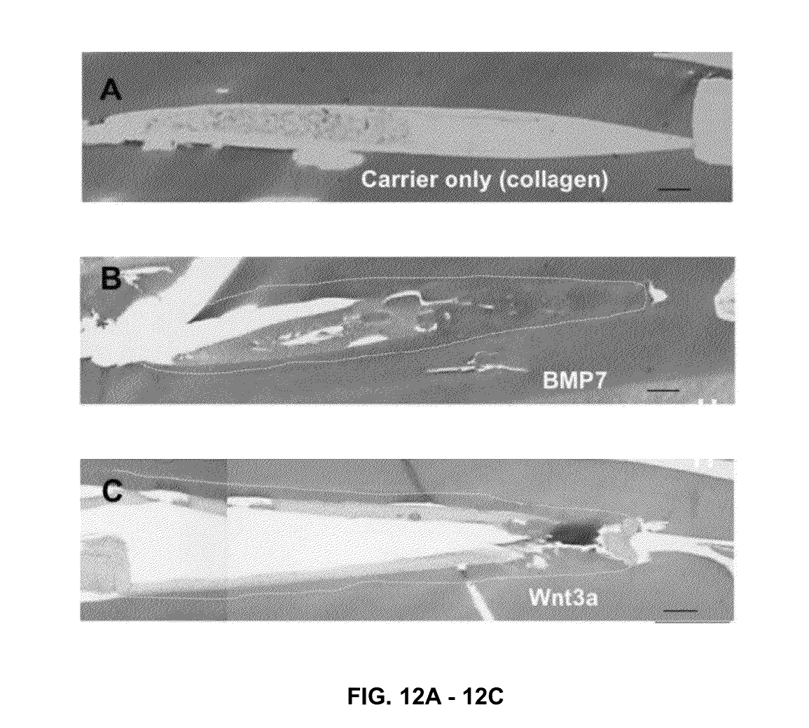

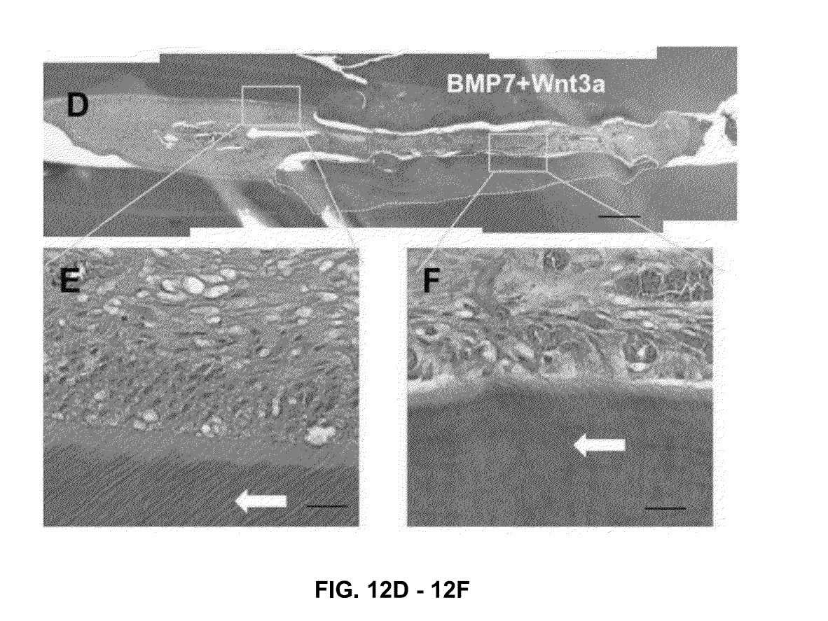

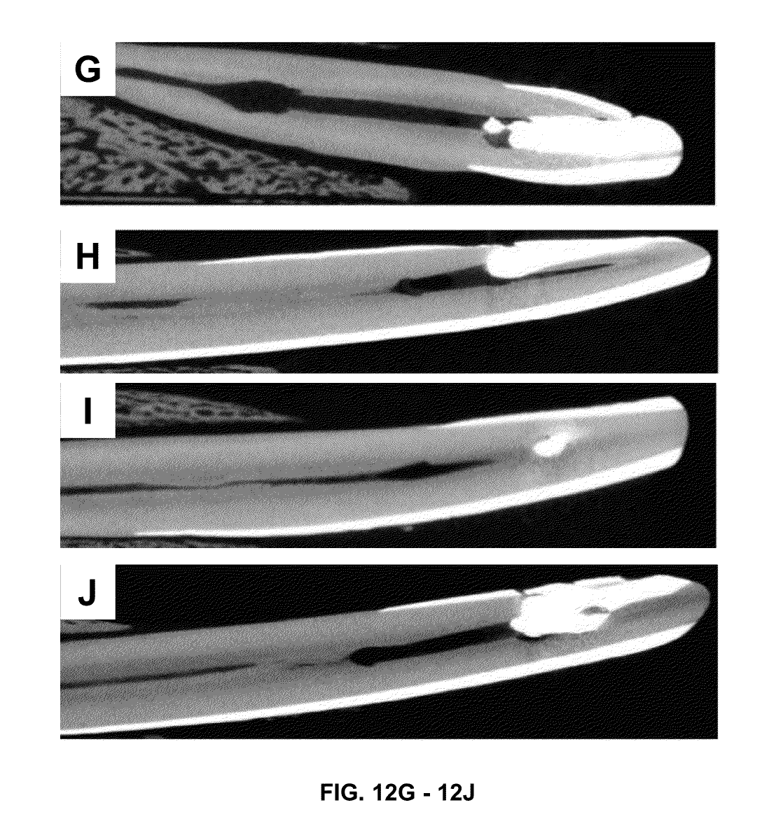

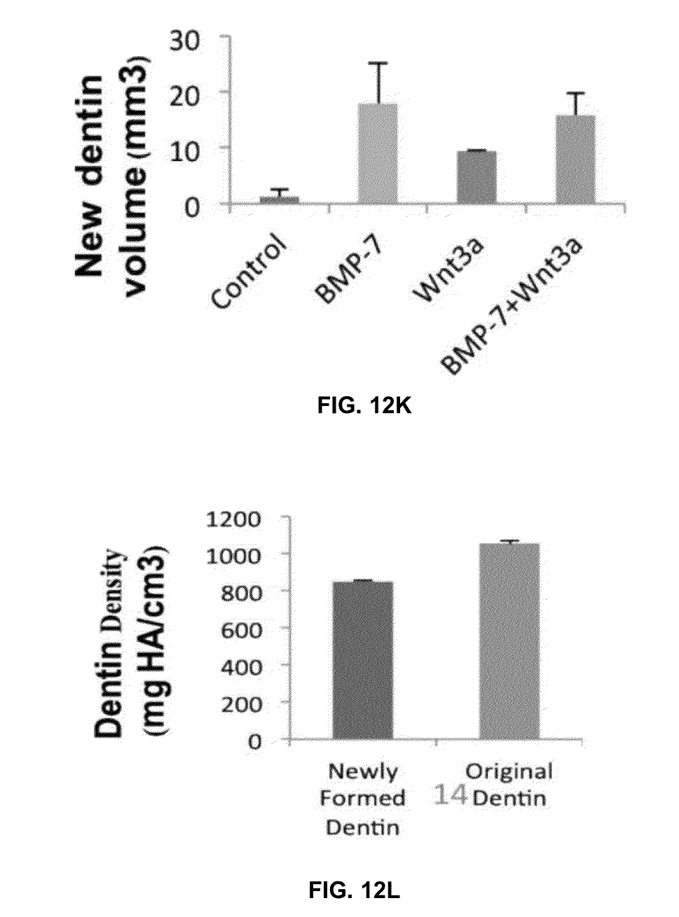

Microspheres of poly-d-l-lactic-co-glycolic acid (PLGA, Sigma, St. Louis, Mo.) of 50:50 PLA/PGA ratio were chosen due to published findings on the cumulative release profile (Moioli et al., 2006; 2007a,b; Clark et al., 2007) (FIG. 3).

One hundred mL of 0.1% PVA was first prepared and put under continuous stirring for 30 minutes at 450 rpm before introducing any other constituent. The 50:50 ratio was prepared using the double emulsion technique ([water-in-oil]-in-water) (Moioli et al., 2006; 2007a,b; Clark et al., 2007). A total of 0.25 g of PLGA was fully dissolved in 1 mL of dichloromethane and emulsified (max vortex speed) with 2.5 μg of recombinant human BMP-7 or NGF diluted in 50 μL solution for 1 minute (water-in-oil). The primary emulsion was then vortexed with 2 mL of 1% polyvinyl alcohol (PVA, 30,000-70,000 MW) for 1 minute ([water-in-oil]-in-water). This mixture was then added to the stirring 0.1% PVA and stirred for 1 minute. A total of 100 mL of 2% isopropanol was added to the final emulsion and continuously stirred for 2 hours under the chemical hood to remove the solvent. PLGA microspheres containing the cytokines were isolated using filtration (2 μm filter), washed with distilled water and frozen in liquid nitrogen for 30 minutes and lyophilized for 48 hours. Freeze-dried PLGA microspheres were stored at −20° C. prior to use.

Both groups of odontogenic and neurotropic cytokine-encapsulated PLGA microspheres were distributed to 4 samples (n=4) each. Each group had 10 mg of the encapsulated cytokines in 1 mL of 1% BSA solution and was continuously agitated on shaker at 37° C. Data points were taken by collecting the entire amount of supernatants weekly for 4-6 weeks. 1 mL of 1% BSA solution was replaced after each collection. The amount of BMP-7 and NGF was quantitatively measured by using the BMP-7 ELISA kit and NGF ELISA kit for each sample.

Results showed that BMP-7 and NGF microspheres were released up to 30-44 days in vitro with the 50:50 ratio of PLA/PGA. A burst-like release was found during the first week and showed similar release profiles compared to previously published results for TGFβ3 controlled release (FIG. 3). Both release profiles showed that 50:50 PLGA could encapsulate BMP-7 and NGF and have similar degradation rates as other previous encapsulated bioactive ingredients.

TABLE 1
Release of BMP-7 over time - ELISA data

| BMP-7 | | | | | BMP-7 | |
|---|---|---|---|---|---|---|
| Release | | | | | Released/ | Cumulative |
| Time | Sample | Sample | Sample | Sample | wk | Release |
| (days) | A | B | C | D | (ng/mL) | (ng/mL) |
| 0 | 0 | 0 | 0 | 0 | 0 | 0 |
| 3 | 0.072 | 0.073 | 0.074 | 0.077 | 0.074 | 0.074 |
| 9 | 0.05 | 0.055 | 0.058 | 0.05 | 0.05325 | 0.12725 |
| 16 | 0.042 | 0.055 | 0.042 | 0.039 | 0.0445 | 0.17175 |
| 23 | 0.071 | 0.083 | 0.074 | 0.073 | 0.07525 | 0.247 |
| 30 | 0.057 | 0.08 | 0.055 | 0.045 | 0.05925 | 0.30625 |
| 44 | 0.032 | 0.029 | 0.033 | 0.03 | 0.031 | 0.33725 |

TABLE 2
NGF ELISA data showing weekly and cumulative release from
PLGA microspheres

| NGF | | | | | NGF | |
|---|---|---|---|---|---|---|
| Release | | | | | Released/ | Cumulative |
| Time | Sample | Sample | Sample | Sample | wk | Release |
| (days) | A | B | C | D | (ng/mL) | (ng/mL) |
| 0 | 0 | 0 | 0 | 0 | 0 | 0 |
| 3 | 1.921 | 2.174 | 2.907 | 1.374 | 2.094 | 2.094 |
| 9 | 2.348 | 1.809 | 2.972 | 0.718 | 1.96175 | 4.05575 |
| 16 | 0.964 | 1.56 | 2.332 | 0.39 | 1.3115 | 5.36725 |
| 23 | 0.618 | 1.284 | 1.332 | 0.703 | 0.98425 | 6.3515 |
| 30 | 0.47 | 1.112 | 0.864 | 0.585 | 0.75775 | 7.10925 |

### Example 3

Primary odontoblasts (Od) were isolated from dental pulp of postnatal day 14 Col1a1(2.3 kb)-GFP mouse incisors and separated from non-odontoblast cells by GFP sorting. Primary osteoblasts (Ob) were isolated from calvaria in the same mice. Global gene expression profiles were analyzed by Alilgent-mouse-genome-oligo microarray. Target genes that may distinguish between odontoblasts (Od) and osteoblasts (Ob) were selected using >5 folds and p<0.01 (adjusted), and confirmed by qRT-PCR, immunohistochemistry and in-situ hybridization. Wnt3a and BMP7, which were encoded by corresponding genes from microarray data, were encapsulated in collagen gel and delivered into endodontically treated root canals of minipig lower incisors in vivo (N=8). Dental pulp regeneration was evaluated 3 months later.

TABLE 3
Selected genes that are differentially expressed.
Bold: highly expressed genes in odontoblasts. Non-
bold: highly expressed genes in osteoblasts.

| | | | Description and |
|---|---|---|---|
| Genes | Access # | Folds | putative function |
| Wnt 10a | NM_009518 | 126 | Activate canonical |
| | | | Wnt/beta-catenin |
| | | | signaling, upstream |
| | | | of DSP |
| Alx3 | NM_007441 | 51 | Patterning of |
| | | | mesoderm during |
| | | | development |
| Pax9 | NM_011041 | 40 | Plays a role in the |
| | | | absence of wisdom |
| | | | teeth in some |
| | | | human populations |
| Dlx1 | NM_010053 | 33 | Development of the |
| | | | ventral forebrain. |
| | | | May play a role in |
| | | | craniofacial patterning and |
| | | | morphogenesis |
| BMP7 | NM_007557 | 30 | Promote osteo/odontogenesis |
| Foxq1 | NM_008239 | 28 | Embryonic |
| | | | development, cell |
| | | | cycle regulation, |
| | | | tissue-specific gene expression |
| Tinagl1 | NM_023476 | 28 | Implicate in the |
| | | | adrenocortical |
| | | | zonation |
| | | | development |
| Lhx8 | NM_010713 | 25 | Differentiation of |
| | | | certain neurons and |
| | | | mesenchymal cells |
| Sox11 | NM_009234 | 17 | Probably important |
| | | | in the developing |
| | | | nervous system |
| GDF10 | NM_145741 | 200 | Positive regulator of |
| | | | osteoblast |
| | | | differentiation |
| Wnt 16 | NM_053116 | 97 | Associate with bone |
| | | | mineral density |
| RANKL | NM_011613 | 25 | Bone remodeling |
| Sox 6 | NM_011445 | 18 | Promote osteoblast |
| | | | differentiation |

Results showed that Col1a1 (2.3 kb) promoter specifically drove GFP expression in Od and Ob. Od accounted for about 2% of total pulp cells. Microarray analysis showed robust expression of 341 genes in Od including wnt family genes, bmp7, dsp, dlx and pax9, and 196 genes in Ob. Wnt3a was shown to be necessary and sufficient to promote odontoblastic differentiation of mesenchyme stem/progenitor cells from alveolar bone marrow and periodontal ligament. Wnt3a was also shown to promote cell migration. In vivo delivery of Wnt3a regenerated not only vascularized pulp tissue in endodontically treated root canals of minipig mandibular incisors, but also neodentin with polarized odontoblasts on native tubular dentin surface, by recruitment of host endogenous cells.

Thus is demonstrated that canonical Wnt signaling pathway is pivotal for odontoblast specification or differentiation from progenitor cells, such as mesenchymal stem cells, and orchestrate dental pulp or dentin regeneration as a microencapsulated, translatable medical device free of cell transplantation.

### Example 4

### Biomaterial Dental Pulp Preservation

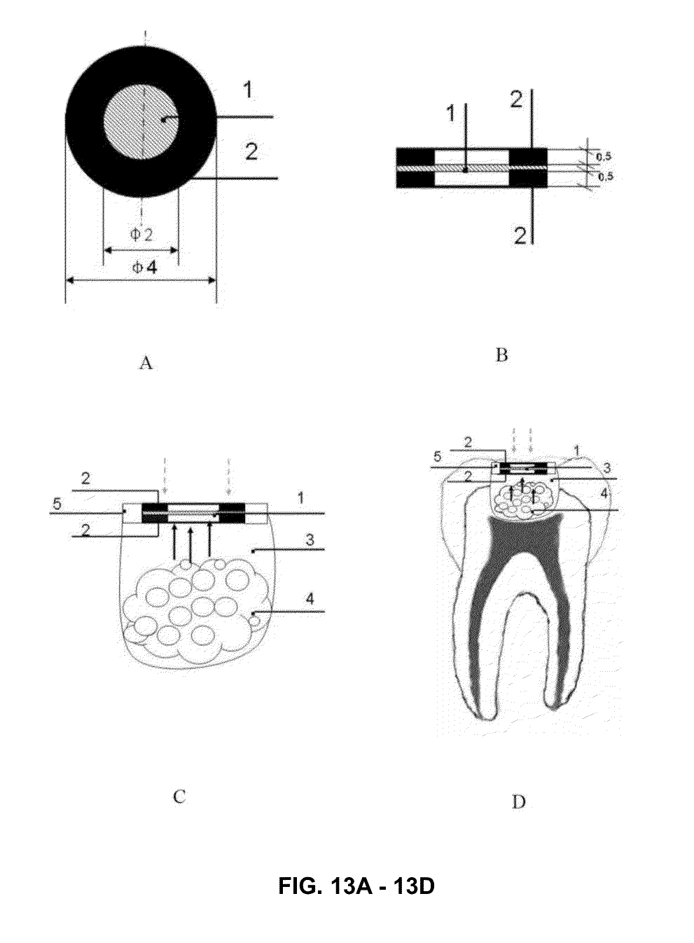

A biomaterial (e.g., an alginate hydrogel) is placed over the surgically prepared tooth cavity or into pulp chamber after removing pathological tissues, including decayed enamel and dentin (see e.g., FIG. 13A-D). For biomaterials such as alginate hydrogel that are placed into the prepared tooth cavity, blood, tissue fluid and water will be absorbed into the biomaterial. The biomaterial device can be removed after 24-48 hours, followed by sealing of the pulp chamber and restoration of the diseased tooth.

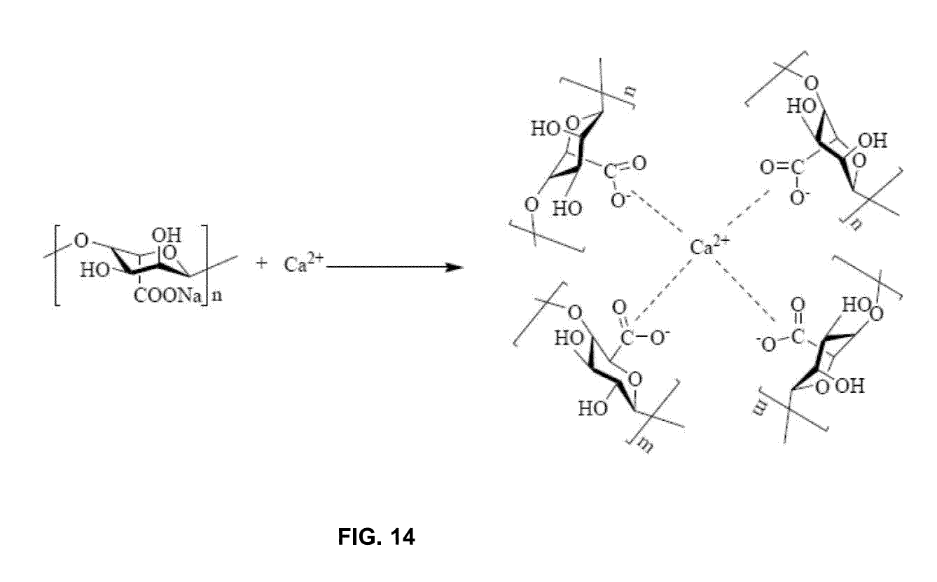

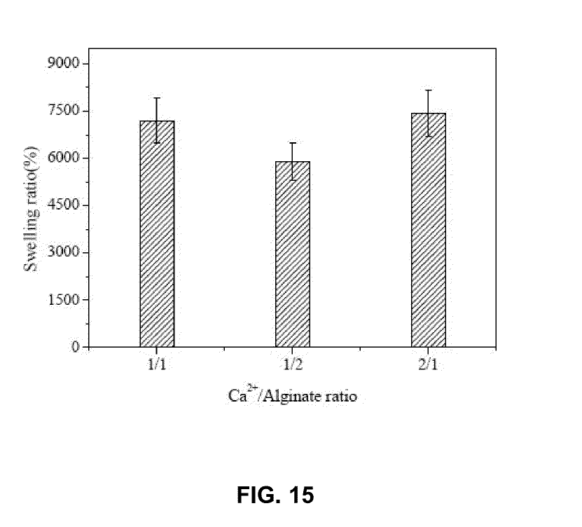

During cross linking, calcium forms stable complex with alginate to form networks (see e.g., FIG. 14). The alginate hydrogels can retain 60˜75 times water of its original weight (see e.g., FIG. 15).

### Example 5

### Endodontic Treatment in Dentally Mature Minipigs

The following example shows endodontic treatment in dentally mature minipigs. Specifically shown in hydrogel mediated dental pulp and dentin regeneration with BMP7 or Wnt3a as a supplement.

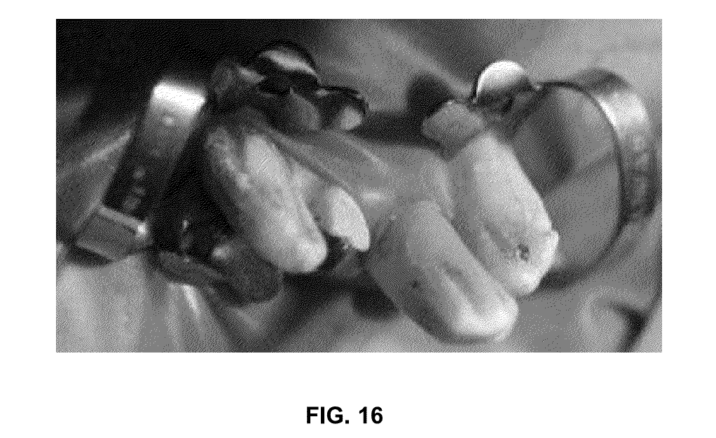

Under anesthesia, a rubber dam was placed to isolate incisors of minipigs, which is a standard of care in endodontic patients (see e.g., FIG. 16. The pulp chamber was accessed by mechanical opening with high speed bur. The coronal and apical pulp were extirpated, followed by instrumentation of root canal dentin, as in endodontic patients. Following disinfection and drying with paper points, hydrogel was delivered by injecting into the root canal and pulp chamber with or without BMP7 (200 ng/ml) or Wnt3a (10 ng/ml). Cavit and composite resin were used to restore the coronal opening as in endodontic patients.

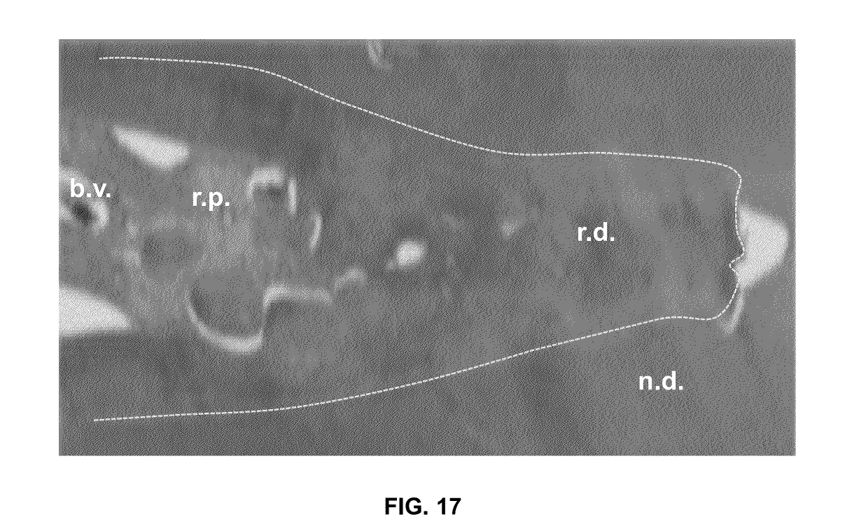

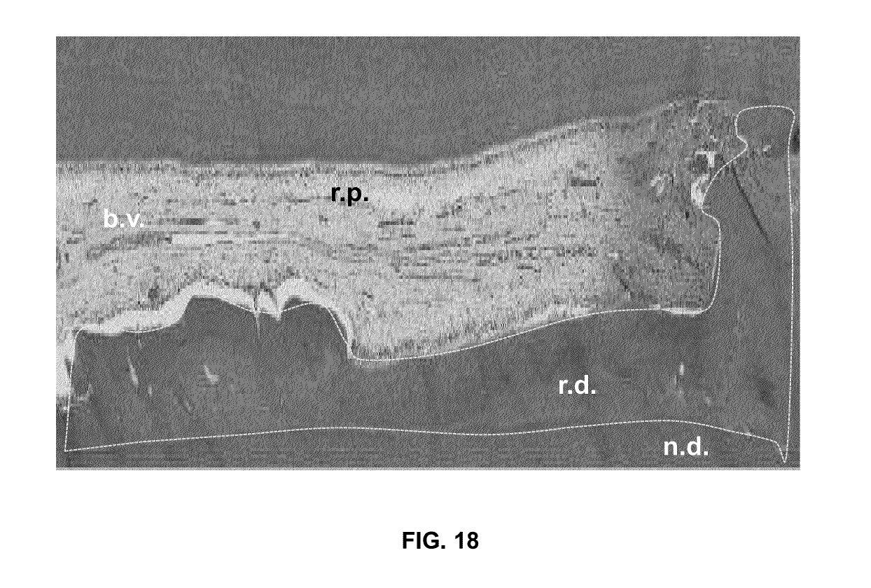

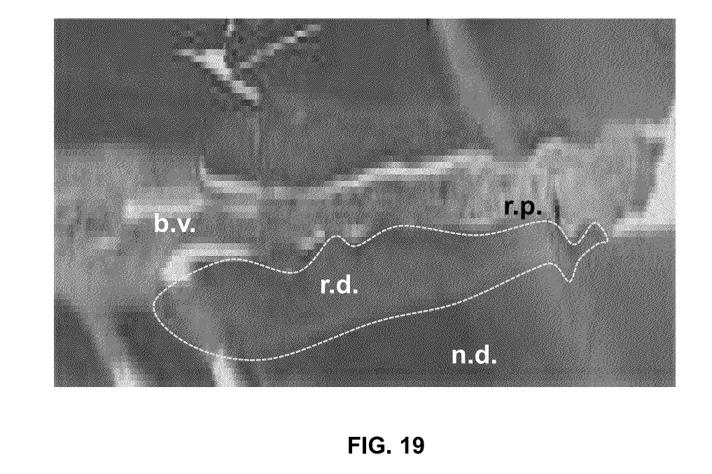

Results for a hydrogel containing BMP7 (200 ng/ml) after four weeks showed that dental pulp and dentin were regenerated (see e.g., FIG. 17). Results for a hydrogel containing Wnt3a protein (10 ng/ml) after four weeks showed that dental pulp and dentin were regenerated (see e.g., FIG. 18). Results for a hydrogel containing BMP7 (200 ng/ml) and Wnt3a protein (10 ng/ml) after four weeks showed that dental pulp and dentin were regenerated (see e.g., FIG. 19).

### REFERENCES

Alhadlaq A and Mao J J. J. Dent. Res. 2003 82:951-956.
Brennan M T, Kent M L, Fox P C, Norton H J, Lockhart P B. The impact of oral disease and nonsurgical treatment on bacteremia in children. J Am. Dent. Assoc. 2007 January; 138(1):80-85.
Christensen, L. R., Møllgård, K., Kjær, I. and Janas, M. S. Immunocytochemical demonstration of nerve bioactive ingredient receptor (NGF-R) in developing human fetal teeth. Anatomy and Embryology Vol 188, Number 3, 247, 1993

Derringer K A et al. Eur. J. Orthodontics 1998 20:357-367.
Edwards P C and J M Mason Head & Face Medicine 2006 2:16.
Fong H K et al. J. Dent. Education 2005 69:555-570.
Goldberg M and Smith A J. Crit. Rev. Oral Biol. Med. 2004 15:13-27.
Hong L and Mao J J. J. Dent. Res. 2004 83:751-756.
Klein O et al. p. 13 in COST Action B23 ORAL FACIAL DEVELOPMENT AND REGENERATION, Joint Meeting of the Working Group 1-4 and the Management Committee, BIOACTIVE INGREDIENTS IN CRANIOFACIAL DEVELOPMENT AND REPAIR, 4-7 May 2006.
Lalani Z et al. J. Oral Maxillofacial Surg. 2005 63:1500-1508.
Lovschall H et al. Adv. Dent. Res. 2001 15:108-112.
Mao J J, Giannobile W V, Helms J A, Hollister S J, Krebsbach P H, Longaker M T, Shi S. Craniofacial tissue engineering by stem cells. J. Dent. Res. 2006 November; 85(11):966-79.
Mathieu S et al. p. 79 in COST Action B23 ORAL FACIAL DEVELOPMENT AND REGENERATION, Joint Meeting of the Working Group 1-4 and the Management Committee, BIOACTIVE INGREDIENTS IN CRANIOFACIAL DEVELOPMENT AND REPAIR, 4-7 May 2006.
Moioli, E. K., Hong, L., Guardado, J., Clark, P., and Mao, J. J. Sustained release of TGF β from PLGA microspheres and its effect on early osteogenic differentiation of human mesenchymal stem cells. Tissue Engineering Vol. 12, Number 3, 537, 2006

Murray P E et al. Crit. Rev. Oral Biol. Med. 2002 13:509-520.
Murray P E et al. J. Endod. 2007 33:377-390.
Nakashima M and Akamine A. J. Endod. 2005 31:711-718.
Nakashima M and Reddi A H. Nat. Biotechnol. 2003 21:1025-1032.
Salvi G E, Siegrist Guldener B E, Amstad T, Joss A, Lang N P. Clinical evaluation of root filled teeth restored with or without post-and-core systems in a specialist practice setting. Int Endod J. 2007 March; 40(3):209-15.
Shay K. Infectious complications of dental and periodontal diseases in the elderly population. Clin Infect Dis. 2002 May 1; 34(9):1215-23.
Stosich M S and Mao J J. Plast. Reconstr. Surg. 2007 119:71-83.
Wang, Y. H., Rutherford, B., Upholt, W. B. and Mina, M. Effects of BMP-7 on mouse tooth mesenchyme and chick mandibular mesenchyme. Developmental Dynamics Vol 216, Issue 4/5, 320, 2000.
Young C. S. et al. J. Dent. Res. 2002 81:695-700.
U.S. Pat. No. 5,885,829.
U.S. Patent Application Publication No. 20050079470

SEQUENCES

```
SEQ ID NO: 1
Wnt3a polypeptide (human)
UniParc P56704
                10 20 30 40 50 60
MAPLGYFLLL CSLKQALGSY PIWWSLAVGP QYSSLGSQPI LCASIPGLVP KQLRFCRNYV
                70 80 90 100 110 120
EIMPSVAEGI KIGIQECQHQ FRGRRWNCTT VHDSLAIFGP VLDKATRESA FVHAIASAGV
              130 140 150 160 170 180
AFAVTRSCAE GTAAICGCSS RHQGSPGKGW KWGGCSEDIE FGGMVSREFA DARENRPDAR
              190 200 210 220 230 240
SAMNRHNNEA GRQAIASHMH LKCKCHGLSG SCEVKTCWWS QPDFRAIGDF LKDKYDSASE
              250 260 270 280 290 300
MVVEKHRESR GWVETLRPRY TYFKVPTERD LVYYEASPNF CEPNPETGSF GTRDRTCNVS
              310 320 330 340 350
SHGIDGCDLL CCGRGHNARA ERRREKCRCV FHWCCYVSCQ ECTRVYDVHT CK
SEQ ID NO: 2
Wnt3a polypeptide (human)
UniParc Q3SY79
                10 20 30 40 50 60
MAPLGYFLLL CSLKQALGSY PIWWSLAVGP QYSSLGSQPI LCASIPGLVP KQLRFCRNYV
                70 80 90 100 110 120
EIMPSVAEGI KIGIQECQHQ FRGRRWNCTT VHDSLAIFGP VLDKATRESA FVHAIASAGV
              130 140 150 160 170 180
AFAVTRSCAE GTAAICGCSS RHQGSPGKGW KWGGCSEDIE FGGMVSREFA DARENRPDAR
              190 200 210 220 230 240
SAMNRHNNEA GRQAIASHMH LKCKCHGLSG SCEVKTCWWS QPDFRAIGDF LKDKYDSASE
              250 260 270 280 290 300
MVVEKHRESR GWVETLRPRY TYFKVPTERD LVYYEASPNF CEPNPETGSF GTRDRTCNVS
              310 320 330 340 350 360
SHGIDGCDLL CCGRGHNARA ERRREKCRCV FHWCCYVSCQ ECTRVYDVHT CKNPGSRAGN
              370 380
SAHQPPHPQP PVRFHPPLRR AGKVP
SEQ ID NO: 3
Wnt3a polypeptide (mouse)
Recombinant full length protein Mouse Wnt3a from cell culture.
SwissProtID = P27467
SLAVGPQYSS LSTQPILCAS IPGLVPKQLR FCRNYVEIMP
SVAEGVKAGI QECQHQFRGR RWNCTTVSNS LAIFGPVLDK ATRESAFVHA
IASAGVAFAV TRSCAEGSAA ICGCSSRLQG SPGEGWKWGG CSEDIEFGGM
VSREFADARE NRPDARSAMN RHNNEAGRQA IASHMHLKCK CHGLSGSCEV
KTCWWSQPDF RTIGDFLKDK YDSASEMVVE KHRESRGWVE TLRPRYTYFK
VPTERDLVYY EASPNFCEPN PETGSFGTRD RTCNVSSHGI DGCDLLCCGR
GHNARTERRR EKCHCVFHWC CYVSCQECTR VYDVHTCK
SEQ ID NO: 4
Wnt3a polypeptide (H. vulgaris)
Recombinant full length Wnt3a protein from Hydra vulgaris, expressed in
E. coli (UniProt Accession Q9GTJ9)
MQYKLALNGK TLKGETTTEA VDAATAEKVF KQYANDNGVD
GEWTYDDATK TFTVTELVPR GSQLWMALGT QTSAIESRPR SSINKNLCRA
LYLHHYQRTV CLNYTDLMLS VAEGIRLGID ECQVQFKHRK WNCTINEHGT
SVFGPIITTA SRESAFISGI ISAGVAFSVT ESCAEGKSVH CRCDNSVRGQ
TDEGWRWGGC NRPITYGIWF SQLFIDQVEK IVKKRKDPRK IMNLHNNKAG
REVIKNLLQT ECKCHGTSGN CNLKTCWRSQ PHFSEIGKIL KEKYDSAHEM
EFLYKVKANG ERKIKDLIPK YKEYLPPSSL DFIYYEESPN YCVKNETLGI
AGTKGRSCNI TSSGVDGCEL MCCQRGYNVN IVQKTHSCEC KFVWCCKVSC
NSCIKMTPEY TCKLVPRGSL EHHHHHH
```

What is claimed is:

1. A composition for regenerating dental tissue comprising:
a matrix or scaffold comprising a hydrogel; and
a therapeutically effective amount of Wnt3a polypeptide; and
a therapeutically effective amount of bone morphogenetic protein 7 (BMP-7); and
optionally, a therapeutically effective amount of vascular endothelial growth factor (VEGF), basic fibroblast growth factor (bFGF), or nerve growth factor (NGF);
wherein
the composition does not comprise a living cell, and
matrix or scaffold comprises Wnt3a and BMP-7 and, optionally, VEGF, bFGF, or NGF.

2. A method for regenerating dental tissue comprising:
providing a composition according to claim 1; and
inserting the composition into a natural or artificial cavity or chamber of a mammalian tooth;
wherein the composition promotes odontoblastic differentiation of a progenitor cell, promotes progenitor cell migration into the dental tissue, promotes angiogenic, odontogenic, fibrogenic, or neurogenic development, so as to regenerate the dental tissue.

3. The method of claim 2, wherein the composition regenerates vascularized pulp tissue or regenerates neodentin.

4. The method of claim 2, wherein the scaffold further comprises a compound selected from the group consisting of platelet-derived growth factor (PDGF), endothelial cell growth factor (ECGF), transforming growth factor-β1 (TGF-β1), epidermal growth factor (EGF), hepatocyte growth factor (HGF), stromal cell-derived factor-1 (SDF1), a bone morphogenetic protein (BMP) other than BMP-7, a TGF-β, a growth and differentiation factor (GDF), insulin-like growth factor-1 (IGF1), a dentin matrix protein, a dentin sialoprotein, a bone sialoprotein, amelogenin, an integrin; an angiogenin; an angiopoietin-1; a del-1; a follistatin; a granulocyte colony-stimulating factor (G-CSF); a hepatocyte growth factor/scatter factor (HGF/SF); an interleukin-8 (IL-8); a leptin; a midkine; a placental growth factor; a platelet-derived endothelial cell growth factor (PD-ECGF); a platelet-derived growth factor-BB (PDGF-BB); a pleiotrophin (PTN); a progranulin; a proliferin; a transforming growth factor-α (TGF-α); a transforming growth factor-β (TGF-β); a tumor necrosis factor-α (TNF-α); a matrix metalloproteinase (MMP); an angiopoietin 1 (ang1); an ang2; a delta-like ligand 4 (DLL4); a connective tissue growth factor (CTGF); a brain derived nerve factor (BDNF); an NT-4; and an NT-3.

5. The method claim 2, wherein the scaffold further comprises an antibiotic or an analgesic.

6. The method of claim 2, wherein the Wnt3a polypeptide, BMP-7, VEGF, bFGF, or NGF is injected into, mixed into, encapsulated in, tethered to, or absorbed in the matrix or scaffold.

7. The method of claim 2, wherein the scaffold has the shape of a human incisor, a human cuspid, a human bicuspid or a human molar, or a natural or artificial cavity or chamber therein.

8. The method of claim 2, wherein the matrix or scaffold comprises:
a natural polymer selected from the group consisting of collagen, gelatin, polysaccharide, chitosan, hydroxyapatite (HA), and polyhydroxyalkanoate;
a synthetic polymer selected from the group consisting of an aliphatic polyester of a poly(α-hydroxy acid), and a polyethylene glycol;
hydroxyapatite; or
an alginate hydrogel.

9. The method of claim 2, wherein the scaffold comprises microchannels having a diameter of (i) between 50 and 500 μm; or (ii) about 200 μm.

10. The method of claim 9, wherein the Wnt3a and BMP-7 and, optionally, VEGF, bFGF, or NGF, are imbedded in a gel in the microchannels of the scaffold.

11. The method of claim 9, wherein the Wnt3a and BMP-7 and, optionally, VEGF, bFGF, or NGF, are encapsulated in a microsphere.

12. The method of claim 2, further comprising making a model of a tooth or tooth cavity using computer aided design (CAD) and synthesizing the scaffold with a bioplotter.

13. The method of claim 2, wherein the Wnt3a polypeptide comprises:
(i) an amino acid sequence of SEQ ID NO: 1, SEQ ID NO: 2, SEQ ID NO: 3, or SEQ ID NO: 4; or
(ii) an amino acid sequence having at least about 95% sequence identity to SEQ ID NO: 1, SEQ ID NO: 2, SEQ ID NO: 3, or SEQ ID NO: 4 and Wnt3a activity.

14. The method of claim 2, wherein the matrix or scaffold is biodegradable.

15. The method of claim 2, wherein Wnt3a is present at a concentration of about 1 ng to about 1,000 mg Wnt3a per ml of solution, where the solution is introduced into the matrix or scaffold.

16. The method of claim 2, wherein BMP-7 is present at a concentration of about 1 ng to about 1,000 mg BMP-7 per ml of solution, where the solution is introduced into the matrix or scaffold.

17. The method of claim 2, wherein Wnt3a is present at a concentration of about 10 ng per ml of solution and BMP-7 is present at a concentration of about 200 ng per ml of solution, where the solution is introduced into the matrix or scaffold.

18. The method of claim 2, wherein
VEGF is present at a concentration of about 1 ng to about 1,000 mg VEGF per ml of solution;
bFGF is present at a concentration of about 1 ng to about 1,000 mg bFGF per ml of solution; or
NGF is present at a concentration of about 1 ng to about 1,000 mg NGF per ml of solution;
where the solution is introduced into the matrix or scaffold.

19. The method of claim 2, further comprising:
exposing traumatized or diseased dental pulp tissue in the tooth pulp chamber or root canal; and
capping or filling at least a portion of a tooth pulp chamber or a root canal with the composition;
wherein de novo vascularized pulp-like tissue forms in the tooth pulp chamber or root canal after capping or filling.

20. The method of claim 19, further comprising:
removing traumatized or diseased dental pulp tissue from the tooth to create a tooth pulp chamber or root canal substantially devoid of traumatized or diseased tissue;
removing substantially all dental pulp tissue from the tooth; or
filling at least a portion of the tooth pulp chamber with an inert material.
